(19) United States
(12) Patent Application Publication
(10) Pub. No.: US 2018/0237744 A1
(43) Pub. Date: Aug. 23, 2018

(54) Placental niche and use thereof to culture stem cells

(71) Applicant: Celularity, Inc., Warren, NJ (US)

(72) Inventor: Mohammad Heidaran, Chatham, NJ (US)

(73) Assignee: CELULARITY INC., Warren, NJ (US)

(21) Appl. No.: 15/717,037

(22) Filed: Sep. 27, 2017

Related U.S. Application Data
(63) Continuation of application No. 11/811,447, filed on Jun. 8, 2007, now abandoned.
(60) Provisional application No. 60/812,366, filed on Jun. 9, 2006.

(51) Int. Cl. C12N 5/073

(52) U.S. Cl. CPC C12N 5/0605; C12N 2500/36; C12N 2500/25; C12N 2501/135; C12N 2501/11; C12N 2533/92; C12N 2501/39

(57) ABSTRACT

The present invention provides methods for culturing, expanding and differentiating stem cells, particularly human embryonic stem cells. The methods comprise culturing the stem cells for a period of time on a collagen biofabric, particularly a collagen biofabric derived from the amniotic membrane, chorion, or both, from mammalian placenta.

This application claims benefit of U.S. Provisional Patent Application No. 60/812,366, filed Jun. 9, 2006, the entirety of which is incorporated by reference herein.

### 1. FIELD OF THE INVENTION

The present invention provides methods of culturing, expanding or differentiating stem cells, particularly human embryonic stem cells, using a placental collagen biofabric. The invention has application, for example, in the areas of cell culture, tissue transplantation, drug discovery and gene therapy.

### 2. BACKGROUND OF THE INVENTION

Human embryonic stem (HES) cells are pluripotent cells that have been derived from the inner cell mass (ICM) of blastocyst stage embryos, or gonadal ridge of embryos. HES cells have the potential to develop into any type of cells and to generate any types of tissues, organs or body parts, including a whole organism. As such, it is expected that the ability to provide normal clonal HES cells on demand and to manipulate the differentiation thereof will provide a powerful tool capable of driving advances in the biomedical, industrial and scientific fields.

Significant hurdles to the practical exploitation of HES cells remain, however. For example, to maintain ES cells in an undifferentiated state, ES cells are usually cultured on feeder cells. However, potential problems in a feeder layer-dependent culture system include: (1) the potential risks of transmission of pathogens from the animal feeder cells to the HES cells and the fact that the current system of propagation (human/animal or human/human co-culture) has been constructed as a xenotransplant, (2) feeder cells come mainly from primary cells, which exhibit significant lot-to-lot variations, making quality control difficult; (3) the limited sources and numbers of feeder cells hamper the mass production and applications of HES cells. Therefore, there is a need for the maintenance and proliferation of undifferentiated HES cells without feeder cells.

Xu et al. (Nat. Biotechnol., 19 (10): 971-974, (2001). WO 03/020920 and U.S. 2003/0017589) were the first to successfully maintain undifferentiated ES cells in a feeder-free culture system. In this system, ES cells are cultured on MATRIGEL™ from the Engelbreth Holm Swarm (EHS) sarcoma or laminin in medium conditioned by mouse embryonic fibroblasts. However, such synthetic matrices and defined-matrix macromolecules are not sufficient to mimic the more complex cell-matrix interactions provided by feeder cells. A study has also indicated that this culture system is only suitable for certain ES cell lines (e.g. H1 and H9), but unsustainable for other ES cell lines (Hovattal et al., Hum. Reprod. 18 (7): 1404-1409, 2003). Moreover, it has recently been appreciated that feeder layer cells can be a source of contamination, for example, by pathogens or non-embryonic or non-human biomolecules, such as the sialic acid Neu5Gc.

Moreover, due to the increasing interest in stem cells from all sources, e.g., placental stem cells, there is a need in the art for improved methods of culturing such cells.

Accordingly, there remains a need in the art for an improved feeder-free culture system for stem cells. The present invention provides such a culture system using a placenta-derived collagen biofabric.

### 3. SUMMARY OF THE INVENTION

The present invention is directed to an improvement in the culture of stem cells, e.g., human embryonic stem cells, placental stem cells, e.g., CD34⁻ or CD34⁺ placental stem cells, comprising culturing the stem cells for a period of time with a collagen biofabric, particularly a collagen biofabric derived from the amniotic membrane, chorion, or both, from mammalian placenta. The collagen biofabric replaces, in some embodiments, feeder cell layers typically used to support stem cells in culture. In other embodiments, the collagen biofabric provides a substrate for the attachment and proliferation of the stem cell.

Without intending to be limited by any theory, it is contemplated that the collagen biofabric of the present invention provides substratum for cell attachment, and, in at least certain embodiments, provides appropriate growth factors supporting the growth of stem cells during culture.

In one aspect, the present invention provides methods for culturing, e.g., expanding, a stem cell comprising culturing the stem cell in a culture medium with a collagen biofabric, wherein said collagen biofabric is derived from a placenta. In a preferred embodiment, said stem cell is exogenous to the collagen biofabric. The stem cell can be cultured under conditions, and for a time, appropriate for survival of a stem cell according to those of skill in the art. In preferred embodiments, the stem cell is an embryonic stem cell or a placental stem cell. In another preferred embodiment, said culturing comprises expansion of said stem cell, or a population of said stem cells. Culturing, e.g., expanding can additionally comprise culturing a somatic cell with the stem cell.

In another aspect, the present invention provides methods for differentiating a stem cell comprising the step of culturing a stem cell in a culture medium with a collagen biofabric in the presence of one or more agents that facilitate or promote the differentiation of the stem cell. Such agents can be, for example, a growth factor, a small molecule, etc. as described herein. In various embodiments, the collagen biofabric comprises one or more such agents, the culture medium in which the stem cell is cultured comprises one or more such agents, or both the collagen biofabric and culture medium comprise one or more such agents.

The stem cell can be cultured under conditions known to those of skill in the art to facilitate the growth of stem cells in cell culture. The stem cell, e.g., a placental stem cell, e.g., a CD34⁻ or CD34⁺ placental stem cell, can be proliferated, or can be differentiated on the collagen biofabric into, for example, a neural cell, an adipocyte, a chondrocyte, an osteocyte, a hepatocyte, a pancreatic cell or a cardiac cell, using appropriate agents that facilitate or promote differentiation into such cells as are well known to those of skill in the art.

Any stem cell can be cultured, expanded or differentiated in accordance with the methods of the invention, including but not limited to, an embryonic stem cell, a placental stem cell (e.g., a CD34⁻ or CD34⁺ placental stem cell), a mesenchymal stem cell, a hematopoietic stem cell, a placental blood- or umbilical cord blood-derived stem cell, a bone marrow-derived stem cell, an adult somatic stem cell, a progenitor cell (e.g., hematopoietic progenitor cell), or cell that is committed to differentiate into a particular cell type. In some embodiments, the stem cell is an embryonic stem cell. In preferred embodiments, the stem cell is a human embryonic stem cell or a placental stem cell. In certain embodiments, the invention also provides a method of culturing a stem cell, wherein the stem cell is not a limbal cell, limbal stem cell, or mesenchymal stem cell from bone marrow.

The collagen biofabric used in the invention is derived from a mammalian placenta. The preferred collagen biofabric is substantially dry, i.e., about 20% or less water by weight. In a specific embodiment, the collagen biofabric is not protease-treated. In another specific embodiment, proteins within said collagen biofabric are not artificially chemically crosslinked, that is, the collagen biofabric is not fixed. In another specific embodiment, the collagen biofabric lacks placental cells, e.g., is decellularized. In another specific embodiment, the collagen biofabric comprises placental cells, e.g., is not decellularized.

The collagen biofabric can comprise hyaluronic acid, e.g., a layer of hyaluronic acid. In a specific embodiment, the hyaluronic acid is crosslinked. In a more specific embodiment, the hyaluronic acid is crosslinked to the collage biofabric.

The collagen biofabric may additionally comprise a bioactive compound not naturally-occurring in the collagen biofabric, or present in a different concentration than in collagen biofabric to which the bioactive compound has not been added. In a more specific embodiment, said bioactive compound is a small organic molecule, an antibiotic, amino acid, pain medication, anti-inflammatory agent, cytokine, growth factor, enzyme inhibitor, kinase inhibitor, an anti-tumor agent, an anti-fungal agent, an anti-viral agent, or an anti-infective agent.

In some embodiments, the collagen biofabric may comprise one or more agents that facilitate or promote the differentiation of stem cells. Such agents are well-known to those of skill in the art, and are described in detail below. The collagen biofabric may also comprise cells endogenous or exogenous to a placenta from which the collagen biofabric is derived. Such cells are described herein.

Any culture medium suitable for culturing, expanding or differentiating stem cells, that is, in which the stem cells proliferate in standard culture conditions for a particular stem cell, known to those of skill in the art can be used in the present invention, appropriate to sources of the cells/tissue from which the stem cell is derived or to which the stem cell will differentiate.

The invention further encompasses the use of a stem cell, a progenitor cell or specific cell type, or populations thereof, wherein the cell or cells are cultured or differentiated according to the methods of the present invention. In certain embodiments, the cell is a neural cell, an adipocyte, an chondrocyte, an osteocyte, a hepatocyte, a pancreatic cell or a cardiac cell made by differentiating a stem cell according to the methods of the present invention.

In some embodiments, the present invention provides for the transplantation of undifferentiated or differentiated stem cells with or without a collagen biofabric produced according to the methods of the present invention to treat or prevent a disease or condition.

The present invention further provides methods of determining the toxicity of a compound to a cell. In some embodiments, the methods comprise culturing a cell with a collagen biofabric under conditions in which the stem cell survives, e.g., proliferates. The cell is then contacted with a compound, and a change of the activity of the cell, for example, in a metabolic parameter of the cell indicating apoptosis, necrosis, or cell death, or a tendency towards apoptosis, necrosis or cell death, is assayed. If a change is detected, as compared to a cell cultured under equivalent conditions and not contacted with the compound, the compound is identified as being toxic to the cell. The cell can be a somatic cell or a stem cell.

In another aspect, the present invention provides methods for determining the effect of a compound on the differentiation of a stem cell by using the collagen biofabric cell culture system of the invention. In some embodiments, the methods comprise culturing said cell with a collagen biofabric under conditions suitable for the differentiation of the cell. The cell is contacted with a compound. The cells are then analyzed for a marker of the differentiation in the presence or absence of the candidate compound. The marker of the differentiation can be a cell surface marker, cell morphology or one or more differentially expressed genes. If a change is identified, said compound is identified as having an effect on the differentiation of said cell.

As used herein, “collagen biofabric” is a substantially flat or sheet-like collagen-containing material derived or obtained from a mammalian amniotic membrane and/or chorion. In a preferred embodiment, collagen biofabric is a decellularized, dehydrated (i.e., 20% or less water by weight) amniotic membrane, chorion, or amniotic membrane and chorion that has not been protease treated, or heat treated above 60° C., substantially as described in Hariri, U.S. Patent Application Publication No. 2004/0048796. In certain embodiments, the collagen biofabric no artificial chemical-induced crosslinks, that is, has not been fixed. The collagen biofabric is typically re-hydrated with, e.g., culture medium prior to culturing, expanding and/or differentiating cells according to the present invention.

### 4. BRIEF DESCRIPTION OF THE FIGURES

FIG. 1 depicts placental stem cells after culture on collagen biofabric (amniotic membrane), collagen, fibronectin, or glass.

### 5. DETAILED DESCRIPTION OF THE INVENTION

The present invention provides methods for culturing, expanding or differentiating stem cells, using a collagen biofabric.

### 5.1. Stem Cells

A stem cell, for example, embryonic stem cell or adult stem cells, can be cultured with collagen biofabric according to the methods of the present invention. As used herein, the term “stem cell” encompasses totipotent, pluripotent and multipotent cells, somatic stem cells or progenitor cells, and the like. Stem cells can be, e.g., placental stem cells (e.g., CD34⁻ or CD34⁺ placental stem cells), umbilical cord stem cells, mesenchymal stem cells, hematopoietic stem cells, placental blood- or umbilical cord blood-derived stem cells, bone marrow-derived stem cells, or somatic stem cells. Somatic stem cells can be, for example, neural stem cells, hepatic stem cells, pancreatic stem cells, endothelial stem cells, cardiac stem cells, muscle stem cells, or epithelial stem cells, skin stem cells, brain stem cells, skin stem cells, endodermal stem cells, ectodermal stem cells, cells described in U.S. Pat. Nos. 5,486,359, 6,261,549 and 6,387,367 (mesenchymal stem cells), and U.S. Pat. No. 5,962,325 (fetal stromal cells). In certain embodiments, the stem cell is not a limbal stem cell.

The stem cells used in the present invention can be derived from, e.g., placenta, umbilical cord, bone marrow, embryo, mesenchyme, neural tissue, pancreatic tissue, muscle tissue (such as cardiac muscle), liver, skin, intestine, nasal epithelium, bone, pancreas, and the like.

In some embodiments, the stem cells used in the present invention are human placental stem cells. Such stem cells are described in, and may routinely be isolated as set forth in, e.g., U.S. Application Publication Nos. 2002/0123141, 2003/0032179, 2003/0180269, 2004/0048796, each of which is incorporated by reference in its entirety.

In some embodiments, the stem cells used in the present invention are embryonic stem cells. Embryonic stem cells can be routinely isolated as described in, e.g., U.S. Pat. Nos. 5,843,780, 6,200,806; and Thomson et al., 1995, Proc. Natl. Acad. Sci. U.S.A. 92:7844. In certain embodiments, the stem cells used in the methods of the invention are human embryonic stem cells. Human embryonic stem cells are described, e.g., in Thomson et al., 1998, Science, 282:1145, and in U.S. Pat. No. 6,200,806.

The present invention also provides for the culture of a stem cell, where the cell is not a bone marrow-derived mesenchymal stem cell or a limbal cell, e.g., limbal stem cell.

Stem cells used in the present invention can be obtained using methods or materials known to those of skill in the art. For instance, stem cells can be obtained from a commercial service, e.g., LifeBank USA (Cedar Knolls, N.J.), ViaCord (Boston Mass.), Cord Blood Registry (San Bruno, Calif.) and Cryocell (Clearwater, Fla.). The stem cells can also be collected using processes or procedures known in the art. Primate primordial stem cells can be obtained, for example, as described in U.S. Pat. Nos. 6,200,806, and 6,800,480. Placental stem cells can be obtained, for example, as described in U.S. Application Publication No. 2003/0032179, the contents of which is incorporated herein by reference in their entireties. Human embryonic stem cells can be obtained from natural sources, such as an embryo, a blastocyst or inner cell mass (ICM) cells, or from a previous or established culture of embryonic cells. Human embryonic stem cells can be prepared from human blastocyst cells using the techniques described by Thomson et al., (U.S. Pat. No. 5,843,780; Science 282:1145, 1998; Curr. Top. Dev. Biol. 38:133 ff., 1998) and Reubinoff et al., 2000, Nature Biotech. 18:399, or U.S. Application Publication No. 2003/0032179, etc. Human embryonic stem cells can also be obtained from the gonadal ridges of human embryo, for example, as described in Reubinoff et al., 2000, Nature, 18:399-404, or U.S. Pat. No. 6,090,622 (human embryonic germ cells), and U.S. Pat. No. 6,562,619, or from frozen embryos, for example, as described in U.S. Pat. No. 6,921,632, or from human placenta, as described in U.S. Application Publication Nos. 2003/0032179, 2002/0123141, 2003/0032179, 2003/0180269, 2004/0048796, the contents of which are hereby incorporated in their entireties.

Stem cells used in the present invention can be from any species known to those of skill in the art. Such stem cells can be, e.g., piscine, avian, reptilian, or mammalian stem cells. Any mammalian stem cells can be used in accordance with the methods of the present invention, including but not limited to stem cells from, e.g., mouse, rat, rabbit, guinea pig, dog, cat, pig, sheep, cow, horse, monkey, human, etc. In certain embodiments, the stem cells are mouse stem cells. In some embodiments, the stem cells are primate stem cells. In preferred embodiments, the stem cells are human stem cells. In particularly preferred embodiment, the stem cells are human embryonic cells.

Stem cells used in the present invention may be used in relatively unpurified form, such as in cord blood or placental blood, or in populations of peripheral blood mononuclear cells obtained by apheresis. The stem cells usable in the present invention may be relatively isolated, i.e., substantially isolated from other cell types. The stem cells can contain a single type of stem cell, or multiple types of stem cells.

Stem cells used in the present invention can be genetically engineered either prior to culturing or during culturing using the collagen biofabric. A polynucleotide can be introduced into the stem cells using any technique known to those of skill in the art, for example, by a viral vector such as an adenoviral or retroviral vector, or by biomechanical means such as liposomal or chemical mediated uptake of the DNA, as described in U.S. Application Publication. No. 2004/0028660, the contents of which are incorporated by reference in their entirety.

### 5.2. Placental Stem Cells

Placental stem cells, e.g., CD34⁻ placental stem cells, referred to hereinafter simply as “placental stem cells,” culturable on collagen biofabric, are stem cells, obtainable from a placenta or part thereof, that adhere to a tissue culture substrate and have the capacity to differentiate into non-placental cell types. Placental stem cells can be either fetal or maternal in origin (that is, can have the genotype of either the mother or fetus). Populations of placental stem cells, or populations of cells comprising placental stem cells, can comprise placental stem cells that are solely fetal or maternal in origin, or can comprise a mixed population of placental stem cells of both fetal and maternal origin. Placental stem cells that can be used in the methods of the present invention are described, e.g., in United States Application Publication Nos. 2005/0019908 and 2003/0180269, the disclosures of which are incorporated herein in their entireties. Placental stem cells, and populations of cells comprising the placental stem cells, can be identified and selected by the morphological, marker, and culture characteristics discussed below.

5.2.1. Physical and Morphological Characteristics

Placental stem cells, when cultured in primary cultures or in cell culture, adhere to the tissue culture substrate, e.g., tissue culture container surface (e.g., tissue culture plastic). Placental stem cells in culture assume a generally fibroblastoid, stellate appearance, with a number of cytoplasmic processes extending from the central cell body. Placental stem cells are, however, morphologically distinguishable from fibroblasts cultured under the same conditions, as the placental stem cells exhibit a greater number of such processes than do fibroblasts. Morphologically, placental stem cells are also differentiable from hematopoietic stem cells, which generally assume a more rounded, or cobblestone, morphology in culture, and embryonic stem cells or embryonic germ cells, which adopt a rounded morphology, whether cultured on a feeder layer or on a substrate, e.g., MATRIGEL™.

Typically, in culture, placental stem cells develop clusters of cells, referred to as embryoid-like bodies, that resemble the embryoid bodies that develop in cultures of embryonic stem cells.

5.2.2. Cell Surface, Molecular and Genetic Markers

Placental stem cells express a plurality of markers that can be used to identify and/or isolate the stem cells. Placental stem cells include stem cells from the whole placenta, or any part thereof (e.g., amnion, chorion, placental cotyledons, umbilical cord, and the like). Placental stem cells are not, however, trophoblasts.

Placental stem cells generally express the markers CD73, CD105, CD200, HLA-G, and/or OCT-4, and generally do not express CD34, CD38, or CD45. Placental stem cells can also express HLA-ABC (MHC-1) and HLA-DR. These markers can be used to identify placental stem cells, and to distinguish placental stem cells from other stem cell types. Because placental stem cells can express CD73 and CD105, they can have mesenchymal stem cell-like characteristics. However, because placental stem cells can express CD200 and HLA-G, a fetal-specific marker, they can be distinguished from mesenchymal stem cells, e.g., bone marrow-derived mesenchymal stem cells, which express neither CD200 nor HLA-G. In the same manner, the lack of expression of CD34, CD38 and/or CD45 identifies the placental stem cells as non-hematopoietic stem cells. In certain embodiments, the placental stem cells are negative for SSEA3 and/or SSEA4. In certain embodiments, the placental stem cells are positive for SSEA3 and/or SSEA4.

Thus, in one embodiment, a placental stem cell is CD200+ or HLA-G±. In a specific embodiment, the stem cell is CD200⁺ and HLA-G⁺. In another specific embodiment, said stem cell is CD73⁺ and CD105⁺. In another specific embodiment, said stem cell is CD34⁻, CD38⁻ or CD45⁻. In another specific embodiment, said stem cell is CD34⁻, CD38⁻ and CD45⁻. In another specific embodiment, said stem cell is CD34⁻, CD38⁻, CD45⁻, CD73⁺ and CD105⁺. In another specific embodiment, said CD200⁺ or HLA-G⁺ stem cell facilitates the formation of embryoid-like bodies in a population of placental cells comprising the stem cells, under conditions that allow the formation of embryoid-like bodies.

A placental stem cell can be selected from a plurality of placental cells by selecting a CD200 or HLA-G placental cell, whereby said cell is a placental stem cell. In a specific embodiment, said selecting comprises selecting a placental cell that is both CD200⁺ and HLA-G⁺. In a specific embodiment, said selecting comprises selecting a placental cell that is also CD73⁺ and CD105⁺. In another specific embodiment, said selecting comprises selecting a placental cell that is also CD34⁻, CD38⁻ or CD45⁻. In another specific embodiment, said selecting comprises selecting a placental cell that is also CD34⁻, CD38⁻ and CD45⁻. In another specific embodiment, said selecting comprises selecting a placental cell that is also CD34⁻, CD38⁻, CD45⁻, CD73⁺ and CD105⁺. In another specific embodiment, said selecting comprises selecting a placental cell that also facilitates the formation of embryoid-like bodies in a population of placental cells comprising the stem cells, under conditions that allow the formation of embryoid-like bodies.

In another embodiment, a placental stem cell is CD73⁺, CD105⁺, and CD200⁺. In another specific embodiment, said stem cell is HLA-G⁺. In another specific embodiment, said stem cell is CD34⁻, CD38⁻ or CD45⁻. In another specific embodiment, said stem cell is CD34⁻, CD38⁻ and CD45⁻. In a more specific embodiment, said stem cell is CD34⁻, CD38⁻, CD45⁻, and HLA-G⁺. In another specific embodiment, the isolated CD73⁺, CD105⁺, and CD200⁺ stem cell facilitates the formation of one or more embryoid-like bodies in a population of placental cells comprising the stem cell, when the population is cultured under conditions that allow the formation of embryoid-like bodies.

A placental stem cell can also be selected from a plurality of placental cells by selecting a CD73⁺, CD105⁺, and CD200⁺ placental cell, whereby said cell is a placental stem cell. In a specific embodiment, said selecting comprises selecting a placental cell that is also HLA-G⁺. In another specific embodiment, said selecting comprises selecting a placental cell that is also CD34⁻, CD38⁻ or CD45⁻. In another specific embodiment, said selecting comprises selecting a placental cell that is also CD34⁻, CD38⁻ and CD45⁻. In another specific embodiment, said selecting comprises selecting a placental cell that is also CD34⁻, CD38⁻, CD45⁻, and HLA-G⁺. In another specific embodiment, said selecting additionally comprises selecting a CD73⁺, CD105⁺, and CD200⁺ stem cell that facilitates the formation of one or more embryoid-like bodies in a population of placental cells comprising the stem cell, when the population is cultured under conditions that facilitate formation of embryoid-like bodies.

In another embodiment, a placental stem cell is CD200⁺ and OCT-4⁺. In a specific embodiment, the stem cell is CD73⁺ and CD105⁺. In another specific embodiment, said stem cell is HLA-G⁺. In another specific embodiment, said stem cell is CD34⁻, CD38⁻ or CD45⁻. In another specific embodiment, said stem cell is CD34⁻, CD38⁻ and CD45⁻. In a more specific embodiment, said stem cell is CD34⁻, CD38⁻, CD45⁻, CD73⁺, CD105⁺ and HLA-G⁺. In another specific embodiment, the stem cell facilitates the production of one or more embryoid-like bodies by a population of placental cells that comprises the stem cell, when the population is cultured under conditions that allow the formation of embryoid-like bodies.

A placental stem cell can also be selected from a plurality of placental cells by selecting a CD200⁺ and OCT-4⁺ placental cell, whereby said cell is a placental stem cell. In a specific embodiment, said selecting comprises selecting a placental cell that is also HLA-G⁺. In another specific embodiment, said selecting comprises selecting a placental cell that is also CD34⁻, CD38⁻ or CD45⁻. In another specific embodiment, said selecting comprises selecting a placental cell that is also CD34⁻, CD38⁻ and CD45⁻. In another specific embodiment, said selecting comprises selecting a placental cell that is also CD34⁻, CD38⁻, CD45⁻, CD73⁺, CD105⁺ and HLA-G⁺. In another specific embodiment, said selecting comprises selecting a placental stem cell that also facilitates the production of one or more embryoid-like bodies by a population of placental cells that comprises the stem cell, when the population is cultured under conditions that allow the formation of embryoid-like bodies.

In another embodiment, a placental stem cell is CD73⁺, CD105⁺ and HLA-G⁺. In another specific embodiment, said stem cell is CD34⁻, CD38⁻ or CD45⁻. In another specific embodiment, said stem cell is CD34⁻, CD38⁻ and CD45⁻. In another specific embodiment, said stem cell is OCT-4⁺. In another specific embodiment, said stem cell is CD200⁺. In a more specific embodiment, said stem cell is CD34⁻, CD38⁻, CD45⁻, OCT-4⁺ and CD200⁺. In another specific embodiment, said stem cell facilitates the formation of embryoid-like bodies in a population of placental cells comprising said stem cell, when the population is cultured under conditions that allow the formation of embryoid-like bodies.

A placental stem cell can also be selected from a plurality of placental cells by selecting a CD73⁺, CD105⁺ and HLA-G⁺ placental cell, whereby said cell is a placental stem cell. In a specific embodiment, said selecting comprises selecting a placental cell that is also CD34⁻, CD38⁻ or CD45⁻. In another specific embodiment, said selecting comprises selecting a placental cell that is also CD34⁻, CD38⁻ and CD45⁻. In another specific embodiment, said selecting comprises selecting a placental cell that is also OCT-4⁺. In another specific embodiment, said selecting comprises selecting a placental cell that is also CD200⁺. In another specific embodiment, said selecting comprises selecting a placental cell that is also CD34⁻, CD38⁻, CD45⁻, OCT-4⁺ and CD200⁺. In another specific embodiment, said selecting comprises selecting a placental cell that also facilitates the formation of one or more embryoid-like bodies in a population of placental cells that comprises said stem cell, when said population is culture under conditions that allow the formation of embryoid-like bodies.

In another embodiment, a placental stem cell is CD73⁺ and CD105⁺ and facilitates the formation of one or more embryoid-like bodies in a population of isolated placental cells comprising said stem cell when said population is cultured under conditions that allow formation of embryoid-like bodies. In a specific embodiment, said stem cell is CD34⁻, CD38⁻ or CD45⁻. In another specific embodiment, said stem cell is CD34⁻, CD38⁻ and CD45⁻. In another specific embodiment, said stem cell is OCT4⁺. In a more specific embodiment, said stem cell is OCT4⁺, CD34⁻, CD38⁻ and CD45⁻.

A placental stem cell can also be selected from a plurality of placental cells by selecting a CD73⁺ and CD105⁺ placental cell that facilitates the formation of one or more embryoid-like bodies in a population of isolated placental cells comprising said stem cell when said population is cultured under conditions that allow formation of embryoid-like bodies, whereby said cell is a placental stem cell. In a specific embodiment, said selecting comprises selecting a placental cell that is also CD34⁻, CD38⁻ or CD45⁻. In another specific embodiment, said selecting comprises selecting a placental cell that is also CD34⁻, CD38⁻ and CD45⁻. In another specific embodiment, said selecting comprises selecting a placental cell that is also OCT-4⁺. In another specific embodiment, said selecting comprises selecting a placental cell that is also CD200⁺. In another specific embodiment, said selecting comprises selecting a placental cell that is also CD34⁻, CD38⁻, CD45⁻, OCT-4⁺ and CD200⁺.

In another embodiment, a placental stem cell is OCT-4⁺ and facilitates formation of one or more embryoid-like bodies in a population of isolated placental cells comprising said stem cell when cultured under conditions that allow formation of embryoid-like bodies. In a specific embodiment, said stem cell is CD73⁺ and CD105⁺. In another specific embodiment, said stem cell is CD34⁻, CD38⁻, or CD45⁻. In another specific embodiment, said stem cell is CD200⁺. In a more specific embodiment, said stem cell is CD73⁺, CD105⁺, CD200⁺, CD34⁻, CD38⁻, and CD45⁻.

A placental stem cell can also be selected from a plurality of placental cells, e.g., by selecting an OCT-4⁺ placental cell that facilitates the formation of one or more embryoid-like bodies in a population of isolated placental cells comprising said stem cell when said population is cultured under conditions that allow formation of embryoid-like bodies, whereby said cell is a placental stem cell. In a specific embodiment, said selecting comprises selecting a placental cell that is also CD34⁻, CD38⁻ or CD45⁻. In another specific embodiment, said selecting comprises selecting a placental cell that is also CD34⁻, CD38⁻ and CD45⁻. In another specific embodiment, said selecting comprises selecting a placental cell that is also CD200⁺. In another specific embodiment, said selecting comprises selecting a placental cell that is also CD200⁺. In another specific embodiment, said selecting comprises selecting a placental cell that is also CD73⁺, CD105⁺, CD200⁺, CD34⁻, CD38⁻, and CD45⁻.

In another embodiment, placental stem cells culturable or differentiable on collagen biofabric are CD10⁺, CD34⁻, CD105⁺, and CD200⁺. An isolated population of placental stem cells can comprise, e.g., at least about 70%, at least about 80%, at least about 90%, at least about 95% or at least about 99% of said placental stem cells. In a specific embodiment of the above embodiments, said stem cells are additionally CD90⁺ and CD45⁻. In a specific embodiment, said stem cell or population of placental stem cells is isolated away from placental cells that are not stem cells. In another specific embodiment, said stem cell or population of placental stem cells is isolated away from placental stem cells that do not display these characteristics. In another specific embodiment, said isolated placental stem cell is non-maternal in origin. In another specific embodiment, at least about 90%, at least about 95%, or at least about 99% of said cells in said isolated population of placental stem cells, are non-maternal in origin.

In another embodiment, placental stem cells culturable or differentiable on collagen biofabric are HLA-A,B,C⁻, CD45⁻, CD133⁻ and CD34⁻. An isolated population of placental stem cells can comprise, e.g., at least about 70%, at least about 80%, at least about 90%, at least about 95% or at least about 99% placental stem cells that are HLA-A,B,C⁻, CD45⁻, CD133⁻ and CD34⁻. In a specific embodiment, said stem cell or population of placental stem cells is isolated away from placental cells that are not stem cells. In another specific embodiment, said population of placental stem cells is isolated away from placental stem cells that do not display these characteristics. In another specific embodiment, said isolated placental stem cell is non-maternal in origin. In another specific embodiment, at least about 90%, at least about 95%, or at least about 99% of said cells in said isolated population of placental stem cells, are non-maternal in origin. In another embodiment, the placental stem cells are isolated from placental perfusate.

In another embodiment, placental stem cells culturable or differentiable on collagen biofabric are CD10⁺, CD13⁺, CD33⁺, CD45⁻, CD117⁻ and CD133⁻. An isolated population of placental stem cells can comprise, e.g., at least about 70%, at least about 80%, at least about 90%, at least about 95% or at least about 99% of placental stem cells that are CD10⁺, CD13⁺, CD33⁺, CD45⁻, CD117⁻ and CD133⁻. In a specific embodiment, said stem cell or population of placental stem cells is isolated away from placental cells that are not stem cells. In another specific embodiment, said isolated placental stem cell is non-maternal in origin. In another specific embodiment, at least about 90%, at least about 95%, or at least about 99% of said cells in said isolated population of placental stem cells, are non-maternal in origin. In another specific embodiment, said stem cell or population of placental stem cells is isolated away from placental stem cells that do not display these characteristics. In another embodiment, the placental stem cells are isolated from placental perfusate.

In another embodiment, placental stem cells culturable or differentiable on collagen biofabric are CD10⁻, CD33⁻, CD44⁺, CD45⁻, and CD11T. An isolated population of placental stem cells can comprise, e.g., at least about 70%, at least about 80%, at least about 90%, at least about 95% or at least about 99% placental stem cells that are CD10⁻, CD33⁻, CD44⁺, CD45⁻, and CD117⁻. In a specific embodiment, said stem cell or population of placental stem cells is isolated away from placental cells that are not stem cells. In another specific embodiment, said isolated placental stem cell is non-maternal in origin. In another specific embodiment, at least about 90%, at least about 95%, or at least 99% of said cells in said isolated population of placental stem cells, are non-maternal in origin. In another specific embodiment, said stem cell or population of placental stem cells is isolated away from placental stem cells that do not display these characteristics. In another embodiment, the placental stem cells are isolated from placental perfusate.

In another embodiment, placental stem cells culturable or differentiable on collagen biofabric are CD10⁻, CD13⁻, CD33⁻, CD45⁻, and CD117⁻. An isolated population of such placental stem cells can comprise, e.g., at least about 70%, at least about 80%, at least about 90%, at least about 95% or at least about 99% placental stem cells that are CD10⁻, CD13⁻, CD33⁻, CD45⁻, and CD117⁻. In a specific embodiment, said stem cell or population of placental stem cells is isolated away from placental cells that are not stem cells. In another specific embodiment, said isolated placental stem cell is non-maternal in origin. In another specific embodiment, at least about 90%, at least about 95%, or at least 99% of said cells in said isolated population of placental stem cells, are non-maternal in origin. In another specific embodiment, said stem cell or population of placental stem cells is isolated away from placental stem cells that do not display these characteristics. In another embodiment, the placental stem cells are isolated from placental perfusate.

In another embodiment, placental stem cells culturable or differentiable on collagen biofabric are HLA A,B,C⁻, CD45⁻, CD34⁻, CD133⁻, positive for CD10, CD13, CD38, CD44, CD90, CD105, CD200 and/or HLA-G, and/or negative for CD117. In another embodiment, the stem cells, or isolated population of placental stem cells, comprises stem cells are HLA A,B,C⁻, CD45⁻, CD34⁻, CD133⁻, and at least about 20%, 25%, 30%, 35%, 40%, 45%, 50%, 55%, 60%, 65%, 70%, 75%, 80%, 85%, 90%, 95%, 98% or about 99% of the stem cells in the population are positive for CD10, CD13, CD38, CD44, CD90, CD105, CD200 and/or HLA-G, and/or negative for CD117. In a specific embodiment, said stem cell or population of placental stem cells is isolated away from placental cells that are not stem cells. In another specific embodiment, said isolated placental stem cell is non-maternal in origin. In another specific embodiment, at least about 90%, at least about 95%, or at least about 99%, of said cells in said isolated population of placental stem cells, are non-maternal in origin. In another specific embodiment, said stem cell or population of placental stem cells is isolated away from placental stem cells that do not display these characteristics. In another embodiment, the invention provides a method of obtaining a placental stem cell that is HLA A,B,C⁻, CD45⁻, CD34⁻, CD133⁻ and positive for CD10, CD13, CD38, CD44, CD90, CD105, CD200 and/or HLA-G, and/or negative for CD117, comprising isolating said cell from placental perfusate.

In another embodiment, placental stem cells culturable or differentiable on collagen biofabric are CD200⁺ and CD10⁺, as determined by antibody binding, and CD117⁻, as determined by both antibody binding and RT-PCR. In another embodiment, the placental stem cell is CD10⁺, CD29⁻, CD54⁺, CD200⁺, HLA-G⁺, HLA class I⁻ and β-2-microglobulin⁻. In another embodiment, the placental stem cell displays, or placental stem cells, display, expression of at least one marker that is at least two-fold higher than for a mesenchymal stem cell (e.g., a bone marrow-derived mesenchymal stem cell). In another specific embodiment, said placental stem cell is non-maternal in origin. In another specific embodiment, at least about 90%, at least about 95%, or at least 99%, of cells in an isolated population of placental stem cells, are non-maternal in origin.

In another embodiment, the placental stem cells, or isolated population of placental stem cells, comprise placental stem cells that are positive for aldehyde dehydrogenase (ALDH), as assessed by an aldehyde dehydrogenase activity assay. Such assays are known in the art (see, e.g., Bostian and Betts, Biochem. J., 173, 787, (1978)). In a specific embodiment, said ALDH assay uses ALDEFLUOR® (Aldagen, Inc., Ashland, Oreg.) as a marker of aldehyde dehydrogenase activity. In a specific embodiment, said plurality is between about 3% and about 25% of cells in said population of cells. In another embodiment, the invention provides a population of umbilical cord stem cells, wherein a plurality of said umbilical cord stem cells are positive for aldehyde dehydrogenase, as assessed by an aldehyde dehydrogenase activity assay that uses ALDEFLUOR® as an indicator of aldehyde dehydrogenase activity. In a specific embodiment, said plurality is between about 3% and about 25% of cells in said population of cells. In another embodiment, said population of placental stem cells or umbilical cord stem cells shows at least three-fold, or at least five-fold, higher ALDH activity than a population of bone marrow-derived mesenchymal stem cells having the same number of cells and cultured under the same conditions.

In various embodiments of any of the above placental stem cells, or populations of placental stem cells, the stem cell or population of placental stem cells has been passaged at least 1, 2, 3, 4, 5, 6, 7, 8, 9, 10, 12, 14, 16, 18, or 20 times, or more, or expanded for 1, 2, 3, 4, 5, 6, 7, 8, 9, 10, 12, 14, 16, 18, 20, 22, 24, 26, 28, 30, 32, 34, 36, 38 or 40 population doublings, or more.

In other embodiments, the placental stem cell or stem cells described above express one or more genes at a detectably higher level than a bone marrow-derived mesenchymal stem cell, wherein said one or more genes are one ore more of ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRCSB, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PJP2, RTN1, SERPINB9, ST3GAL6, ST6GALNAC5, SLC12A8, TCF21, TGFB2, VTN, and/or ZC3H12A, and wherein said bone marrow derived stem cell has undergone a number of passages in culture equivalent to the number of passages said placental stem cell has undergone. Sequences corresponding to these genes are found on Affymetrix GENECHIP® arrays. These genes can also be found at GenBank accession nos. NM_001615 (ACTG2), BC065545 (ADARB1), (NM_181847 (AMIGO2), AY358590 (ARTS-1), BC074884 (B4GALT6), BC008396 (BCHE), BC020196 (C11orf9), BC031103 (CD200), NM_001845 (COL4A1), NM_001846 (COL4A2), BC052289 (CPA4), BC094758 (DMD), AF293359 (DSC3), NM_001943 (DSG2), AF338241 (ELOVL2), AY336105 (F2RL1), NM_018215 (FLJ10781), AY416799 (GATA6), BC075798 (GPR126), NM_016235 (GPRC5B), AF340038 (ICAM1), BC000844 (IER3), BC066339 (IGFBP7), BC013142 (IL1A), BT019749 (IL6), BC007461 (IL18), (BC072017) KRT18, BC075839 (KRT8), BC060825 (LIPG), BC065240 (LRAP), BC010444 (MATN2), BC011908 (MEST), BC068455 (NFE2L3), NM_014840 (NUAK1), AB006755 (PCDH7), NM_014476 (PDLIM3), BC126199 (PKP-2), BC090862 (RTN1), BC002538 (SERPINB9), BC023312 (ST3GAL6), BC001201 (ST6GALNAC5), BC126160 or BC065328 (SLC12A8), BC025697 (TCF21), BC096235 (TGFB2), BC005046 (VTN), and BC005001 (ZC3H12A) as of December 2006.

In a more specific embodiment, a placental stem cell or placental stem cells expresses ACTG2, ADARB1, AMIGO2, ARTS-1, B4GALT6, BCHE, C11orf9, CD200, COL4A1, COL4A2, CPA4, DMD, DSC3, DSG2, ELOVL2, F2RL1, FLJ10781, GATA6, GPR126, GPRCSB, HLA-G, ICAM1, IER3, IGFBP7, IL1A, IL6, IL18, KRT18, KRT8, LIPG, LRAP, MATN2, MEST, NFE2L3, NUAK1, PCDH7, PDLIM3, PKP2, RTN1, SERPINB9, ST3GAL6, ST6GALNAC5, SLC12A8, TCF21, TGFB2, VTN, and ZC3H12A at a detectably higher level than a bone marrow-derived mesenchymal stem cell.

Generally, placental stem cells are obtained from a mammalian placenta using a physiologically-acceptable solution, e.g., a stem cell collection composition. A stem cell collection composition is described in detail in related U.S. patent application Ser. No. 11/648,812, entitled “Improved Medium for Collecting Placental Stem Cells and Preserving Organs,” filed on Dec. 28, 2006.

A placental stem cell collection composition can comprise any physiologically-acceptable solution suitable for the collection and/or culture of stem cells, for example, a saline solution (e.g., phosphate-buffered saline, Kreb's solution, modified Kreb's solution, Eagle's solution, 0.9% NaCl. etc.), a culture medium (e.g., DMEM, H.DMEM, etc.), and the like.

A placental stem cell collection composition can comprise one or more components that tend to preserve placental stem cells, that is, prevent the placental stem cells from dying, or delay the death of the placental stem cells, reduce the number of placental stem cells in a population of cells that die, or the like, from the time of collection to the time of culturing. Such components can be, e.g., an apoptosis inhibitor (e.g., a caspase inhibitor or JNK inhibitor); a vasodilator (e.g., magnesium sulfate, an antihypertensive drug, atrial natriuretic peptide (ANP), adrenocorticotropin, corticotropin-releasing hormone, sodium nitroprusside, hydralazine, adenosine triphosphate, adenosine, indomethacin or magnesium sulfate, a phosphodiesterase inhibitor, etc.); a necrosis inhibitor (e.g., 2-(1H-Indo1-3-yl)-3-pentylamino-maleimide, pyrrolidine dithiocarbamate, or clonazepam); a TNF-α inhibitor; and/or an oxygen-carrying perfluorocarbon (e.g., perfluorooctyl bromide, perfluorodecyl bromide, etc.).

A placental stem cell collection composition can comprise one or more tissue-degrading enzymes, e.g., a metalloprotease, a serine protease, a neutral protease, an RNase, or a DNase, or the like. Such enzymes include, but are not limited to, collagenases (e.g., collagenase I, II, III or IV, a collagenase from Clostridium histolyticum, etc.); dispase, thermolysin, elastase, trypsin, LIBERASE, hyaluronidase, and the like.

A placental stem cell collection composition can comprise a bacteriocidally or bacteriostatically effective amount of an antibiotic. In certain non-limiting embodiments, the antibiotic is a macrolide (e.g., tobramycin), a cephalosporin (e.g., cephalexin, cephradine, cefuroxime, cefprozil, cefaclor, cefixime or cefadroxil), a clarithromycin, an erythromycin, a penicillin (e.g., penicillin V) or a quinolone (e.g., ofloxacin, ciprofloxacin or norfloxacin), a tetracycline, a streptomycin, etc. In a particular embodiment, the antibiotic is active against Gram(+) and/or Gram(−) bacteria, e.g., Pseudomonas aeruginosa, Staphylococcus aureus, and the like.

A placental stem cell collection composition can also comprise one or more of the following compounds: adenosine (about 1 mM to about 50 mM); D-glucose (about 20 mM to about 100 mM); magnesium ions (about 1 mM to about 50 mM); a macromolecule of molecular weight greater than 20,000 daltons, in one embodiment, present in an amount sufficient to maintain endothelial integrity and cellular viability (e.g., a synthetic or naturally occurring colloid, a polysaccharide such as dextran or a polyethylene glycol present at about 25 g/l to about 100 g/l, or about 40 g/l to about 60 g/l); an antioxidant (e.g., butylated hydroxyanisole, butylated hydroxytoluene, glutathione, vitamin C or vitamin E present at about 25 μM to about 100 μM); a reducing agent (e.g., N-acetylcysteine present at about 0.1 mM to about 5 mM); an agent that prevents calcium entry into cells (e.g., verapamil present at about 2 μM to about 25 μM); nitroglycerin (e.g., about 0.05 g/L to about 0.2 g/L); an anticoagulant, in one embodiment, present in an amount sufficient to help prevent clotting of residual blood (e.g., heparin or hirudin present at a concentration of about 1000 units/1 to about 100,000 units/1); or an amiloride containing compound (e.g., amiloride, ethyl isopropyl amiloride, hexamethylene amiloride, dimethyl amiloride or isobutyl amiloride present at about 1.0 μM to about 5 μM).

5.2.3. Collection and Handling of Placenta

Generally, a human placenta is recovered shortly after its expulsion after birth. In one embodiment, the placenta is recovered from a patient after informed consent and after a complete medical history of the patient is taken and is associated with the placenta. Preferably, the medical history continues after delivery. Such a medical history can be used to coordinate subsequent use of the placenta or the stem cells harvested therefrom. For example, human placental stem cells can be used, in light of the medical history, for personalized medicine for the infant associated with the placenta, or for parents, siblings or other relatives of the infant.

Prior to recovery of placental stem cells, the umbilical cord blood and placental blood are removed. In certain embodiments, after delivery, the cord blood in the placenta is recovered. The placenta can be subjected to a conventional cord blood recovery process. Typically a needle or cannula is used, with the aid of gravity, to exsanguinate the placenta (see, e.g., Anderson, U.S. Pat. No. 5,372,581; Hessel et al., U.S. Pat. No. 5,415,665). The needle or cannula is usually placed in the umbilical vein and the placenta can be gently massaged to aid in draining cord blood from the placenta. Such cord blood recovery may be performed commercially, e.g., LifeBank Inc., Cedar Knolls, N.J., ViaCord, Cord Blood Registry and Cryocell. Preferably, the placenta is gravity drained without further manipulation so as to minimize tissue disruption during cord blood recovery.

Typically, a placenta is transported from the delivery or birthing room to another location, e.g., a laboratory, for recovery of cord blood and collection of stem cells by, e.g., perfusion or tissue dissociation. The placenta is preferably transported in a sterile, thermally insulated transport device (maintaining the temperature of the placenta between 20-28° C.), for example, by placing the placenta, with clamped proximal umbilical cord, in a sterile zip-lock plastic bag, which is then placed in an insulated container. In another embodiment, the placenta is transported in a cord blood collection kit substantially as described in pending United States patent application publication no. 2006/0060494. Preferably, the placenta is delivered to the laboratory four to twenty-four hours following delivery. In certain embodiments, the proximal umbilical cord is clamped, preferably within 4-5 cm (centimeter) of the insertion into the placental disc prior to cord blood recovery. In other embodiments, the proximal umbilical cord is clamped after cord blood recovery but prior to further processing of the placenta.

The placenta, prior to stem cell collection, can be stored under sterile conditions and at either room temperature or at a temperature of 5 to 25° C. (centigrade). The placenta may be stored for a period of longer than forty eight hours, and preferably for a period of four to twenty-four hours prior to perfusing the placenta to remove any residual cord blood. The placenta is preferably stored in an anticoagulant solution at a temperature of 5 to 25° C. (centigrade). Suitable anticoagulant solutions are well known in the art. For example, a solution of heparin or warfarin sodium can be used. In a preferred embodiment, the anticoagulant solution comprises a solution of heparin (e.g., 1% w/w in 1:1000 solution). The exsanguinated placenta is preferably stored for no more than 36 hours before placental stem cells are collected.

The mammalian placenta or a part thereof, once collected and prepared generally as above, can be treated in any art-known manner, e.g., can be perfused or disrupted, e.g., digested with one or more tissue-disrupting enzymes, to obtain stem cells.

5.2.4. Physical Disruption and Enzymatic Digestion of Placental Tissue

In one embodiment, stem cells are collected from a mammalian placenta by physical disruption, e.g., enzymatic digestion, of the organ. For example, the placenta, or a portion thereof, may be, e.g., crushed, sheared, minced, diced, chopped, macerated or the like, while in contact with the stem cell collection composition of the invention, and the tissue subsequently digested with one or more enzymes. The placenta, or a portion thereof, may also be physically disrupted and digested with one or more enzymes, and the resulting material then immersed in, or mixed into, the stem cell collection composition of the invention. Any method of physical disruption can be used, provided that the method of disruption leaves a plurality, more preferably a majority, and more preferably at least 60%, 70%, 80%, 90%, 95%, 98%, or 99% of the cells in said organ viable, as determined by, e.g., trypan blue exclusion.

The placenta can be dissected into components prior to physical disruption and/or enzymatic digestion and stem cell recovery. For example, placental stem cells can be obtained from the amniotic membrane, chorion, umbilical cord, placental cotyledons, or any combination thereof. Preferably, placental stem cells are obtained from placental tissue comprising amnion and chorion. Typically, placental stem cells can be obtained by disruption of a small block of placental tissue, e.g., a block of placental tissue that is about 1, 2, 3, 4, 5, 6, 7, 8, 9, 10, 20, 30, 40, 50, 60, 70, 80, 90, 100, 200, 300, 400, 500, 600, 700, 800, 900 or about 1000 cubic millimeters in volume.

A preferred stem cell collection composition comprises one or more tissue-disruptive enzyme(s). Enzymatic digestion preferably uses a combination of enzymes, e.g., a combination of a matrix metalloprotease and a neutral protease, for example, a combination of collagenase and dispase. In one embodiment, enzymatic digestion of placental tissue uses a combination of a matrix metalloprotease, a neutral protease, and a mucolytic enzyme for digestion of hyaluronic acid, such as a combination of collagenase, dispase, and hyaluronidase or a combination of LIBERASE (Boehringer Mannheim Corp., Indianapolis, Ind.) and hyaluronidase. Other enzymes that can be used to disrupt placenta tissue include papain, deoxyribonucleases, serine proteases, such as trypsin, chymotrypsin, or elastase. Serine proteases may be inhibited by alpha 2 microglobulin in serum and therefore the medium used for digestion is usually serum-free. EDTA and DNase are commonly used in enzyme digestion procedures to increase the efficiency of cell recovery. The digestate is preferably diluted so as to avoid trapping stem cells within the viscous digest.

Any combination of tissue digestion enzymes can be used. Typical concentrations for tissue digestion enzymes include, e.g., 50-200 U/mL for collagenase I and collagenase IV, 1-10 U/mL for dispase, and 10-100 U/mL for elastase. Proteases can be used in combination, that is, two or more proteases in the same digestion reaction, or can be used sequentially in order to liberate placental stem cells. For example, in one embodiment, a placenta, or part thereof, is digested first with an appropriate amount of collagenase I at 2 mg/ml for 30 minutes, followed by digestion with trypsin, 0.25%, for 10 minutes, at 37° C. Serine proteases are preferably used consecutively following use of other enzymes.

In another embodiment, the tissue can further be disrupted by the addition of a chelator, e.g., ethylene glycol bis(2-aminoethyl ether)-N,N,N′N′-tetraacetic acid (EGTA) or ethylenediaminetetraacetic acid (EDTA) to the stem cell collection composition comprising the stem cells, or to a solution in which the tissue is disrupted and/or digested prior to isolation of the stem cells with the stem cell collection composition.

It will be appreciated that where an entire placenta, or portion of a placenta comprising both fetal and maternal cells (for example, where the portion of the placenta comprises the chorion or cotyledons), the placental stem cells collected will comprise a mix of placental stem cells derived from both fetal and maternal sources. Where a portion of the placenta that comprises no, or a negligible number of, maternal cells (for example, amnion), the placental stem cells collected will comprise almost exclusively fetal placental stem cells.

5.2.5. Placental Perfusion

Placental stem cells can also be obtained by perfusion of the mammalian placenta. Methods of perfusing mammalian placenta to obtain stem cells are disclosed, e.g., in Hariri, U.S. Application Publication No. 2002/0123141, and in related U.S. Provisional Application No. 60/754,969, entitled “Improved Medium for Collecting Placental Stem Cells and Preserving Organs,” filed on Dec. 29, 2005.

Placental stem cells can be collected by perfusion, e.g., through the placental vasculature, using, e.g., a stem cell collection composition as a perfusion solution. In one embodiment, a mammalian placenta is perfused by passage of perfusion solution through either or both of the umbilical artery and umbilical vein. The flow of perfusion solution through the placenta may be accomplished using, e.g., gravity flow into the placenta. Preferably, the perfusion solution is forced through the placenta using a pump, e.g., a peristaltic pump. The umbilical vein can be, e.g., cannulated with a cannula, e.g., a TEFLON® or plastic cannula, that is connected to a sterile connection apparatus, such as sterile tubing. The sterile connection apparatus is connected to a perfusion manifold.

In preparation for perfusion, the placenta is preferably oriented (e.g., suspended) in such a manner that the umbilical artery and umbilical vein are located at the highest point of the placenta. The placenta can be perfused by passage of a perfusion fluid, e.g., the stem cell collection composition of the invention, through the placental vasculature, or through the placental vasculature and surrounding tissue. In one embodiment, the umbilical artery and the umbilical vein are connected simultaneously to a pipette that is connected via a flexible connector to a reservoir of the perfusion solution. The perfusion solution is passed into the umbilical vein and artery. The perfusion solution exudes from and/or passes through the walls of the blood vessels into the surrounding tissues of the placenta, and is collected in a suitable open vessel from the surface of the placenta that was attached to the uterus of the mother during gestation. The perfusion solution may also be introduced through the umbilical cord opening and allowed to flow or percolate out of openings in the wall of the placenta which interfaced with the maternal uterine wall. In another embodiment, the perfusion solution is passed through the umbilical veins and collected from the umbilical artery, or is passed through the umbilical artery and collected from the umbilical veins.

In one embodiment, the proximal umbilical cord is clamped during perfusion, and more preferably, is clamped within 4-5 cm (centimeter) of the cord's insertion into the placental disc.

The first collection of perfusion fluid from a mammalian placenta during the exsanguination process is generally colored with residual red blood cells of the cord blood and/or placental blood. The perfusion fluid becomes more colorless as perfusion proceeds and the residual cord blood cells are washed out of the placenta. Generally from 30 to 100 ml (milliliter) of perfusion fluid is adequate to initially exsanguinate the placenta, but more or less perfusion fluid may be used depending on the observed results.

The volume of perfusion liquid used to collect placental stem cells may vary depending upon the number of stem cells to be collected, the size of the placenta, the number of collections to be made from a single placenta, etc. In various embodiments, the volume of perfusion liquid may be from 50 mL to 5000 mL, 50 mL to 4000 mL, 50 mL to 3000 mL, 100 mL to 2000 mL, 250 mL to 2000 mL, 500 mL to 2000 mL, or 750 mL to 2000 mL. Typically, the placenta is perfused with 700-800 mL of perfusion liquid following exsanguination.

The placenta can be perfused a plurality of times over the course of several hours or several days. Where the placenta is to be perfused a plurality of times, it may be maintained or cultured under aseptic conditions in a container or other suitable vessel, and perfused with the stem cell collection composition, or a standard perfusion solution (e.g., a normal saline solution such as phosphate buffered saline (“PBS”)) with or without an anticoagulant (e.g., heparin, warfarin sodium, coumarin, bishydroxycoumarin), and/or with or without an antimicrobial agent (e.g., ÿ-mercaptoethanol (0.1 mM); antibiotics such as streptomycin (e.g., at 40-100 μg/ml), penicillin (e.g., at 40 U/ml), amphotericin B (e.g., at 0.5 μg/ml). In one embodiment, an isolated placenta is maintained or cultured for a period of time without collecting the perfusate, such that the placenta is maintained or cultured for 1, 2, 3, 4, 5, 6, 7, 8, 9, 10, 11, 12, 13, 14, 15, 16, 17, 18, 19, 20, 21, 22, 23, or 24 hours, or 2 or 3 or more days before perfusion and collection of perfusate. The perfused placenta can be maintained for one or more additional time(s), e.g., 1, 2, 3, 4, 5, 6, 7, 8, 9, 10, 11, 12, 13, 14, 15, 16, 17, 18, 19, 20, 21, 22, 23, 24 or more hours, and perfused a second time with, e.g., 700-800 mL perfusion fluid. The placenta can be perfused 1, 2, 3, 4, 5 or more times, for example, once every 1, 2, 3, 4, 5 or 6 hours. In a preferred embodiment, perfusion of the placenta and collection of perfusion solution, e.g., stem cell collection composition, is repeated until the number of recovered nucleated cells falls below 100 cells/ml. The perfusates at different time points can be further processed individually to recover time-dependent populations of cells, e.g., stem cells. Perfusates from different time points can also be pooled.

Without wishing to be bound by any theory, after exsanguination and a sufficient time of perfusion of the placenta, placental stem cells are believed to migrate into the exsanguinated and perfused microcirculation of the placenta where, according to the methods of the invention, they are collected, preferably by washing into a collecting vessel by perfusion. Perfusing the isolated placenta not only serves to remove residual cord blood but also provide the placenta with the appropriate nutrients, including oxygen. The placenta may be cultivated and perfused with a similar solution which was used to remove the residual cord blood cells, preferably, without the addition of anticoagulant agents.

Perfusion according to the methods of the invention results in the collection of significantly more placental stem cells than the number obtainable from a mammalian placenta not perfused with said solution, and not otherwise treated to obtain stem cells (e.g., by tissue disruption, e.g., enzymatic digestion). In this context, “significantly more” means at least 10% more. Perfusion according to the methods of the invention yields significantly more placental stem cells than, e.g., the number of placental stem cells obtainable from culture medium in which a placenta, or portion thereof, has been cultured.

Stem cells can be isolated from placenta by perfusion with a solution comprising one or more proteases or other tissue-disruptive enzymes. In a specific embodiment, a placenta or portion thereof (e.g., amniotic membrane, amnion and chorion, placental lobule or cotyledon, umbilical cord, or combination of any of the foregoing) is brought to 25-37° C., and is incubated with one or more tissue-disruptive enzymes in 200 mL of a culture medium for 30 minutes. Cells from the perfusate are collected, brought to 4° C., and washed with a cold inhibitor mix comprising 5 mM EDTA, 2 mM dithiothreitol and 2 mM beta-mercaptoethanol. The stem cells are washed after several minutes with a cold (e.g., 4° C.) stem cell collection composition of the invention.

It will be appreciated that perfusion using the pan method, that is, whereby perfusate is collected after it has exuded from the maternal side of the placenta, results in a mix of fetal and maternal cells. As a result, the cells collected by this method comprise a mixed population of placental stem cells of both fetal and maternal origin. In contrast, perfusion solely through the placental vasculature, whereby perfusion fluid is passed through one or two placental vessels and is collected solely through the remaining vessel(s), results in the collection of a population of placental stem cells almost exclusively of fetal origin.

5.2.6. Isolation, Sorting, and Characterization of Placental Stem Cells

Stem cells from mammalian placenta, whether obtained by perfusion or enzymatic digestion, can initially be purified from (i.e., be isolated from) other cells by Ficoll gradient centrifugation. Such centrifugation can follow any standard protocol for centrifugation speed, etc. In one embodiment, for example, cells collected from the placenta are recovered from perfusate by centrifugation at 5000×g for 15 minutes at room temperature, which separates cells from, e.g., contaminating debris and platelets. In another embodiment, placental perfusate is concentrated to about 200 ml, gently layered over Ficoll, and centrifuged at about 1100×g for 20 minutes at 22° C., and the low-density interface layer of cells is collected for further processing.

Cell pellets can be resuspended in fresh stem cell collection composition, or a medium suitable for stem cell maintenance, e.g., IMDM serum-free medium containing 2 U/ml heparin and 2 mM EDTA (GibcoBRL, NY). The total mononuclear cell fraction can be isolated, e.g., using LYMPHOPREP™ (Nycomed Pharma, Oslo, Norway) according to the manufacturer's recommended procedure.

As used herein, “isolating” placental stem cells means to remove at least 20%, 30%, 40%, 50%, 60%, 70%, 80%, 90%, 95% or 99% of the cells with which the stem cells are normally associated in the intact mammalian placenta. A stem cell from an organ is “isolated” when it is present in a population of cells that comprises fewer than 50% of the cells with which the stem cell is normally associated in the intact organ.

Placental cells obtained by perfusion or digestion can, for example, be further, or initially, isolated by differential trypsinization using, e.g., a solution of 0.05% trypsin with 0.2% EDTA (Sigma, St. Louis Mo.). Differential trypsinization is possible because placental stem cells typically detach from plastic surfaces within about five minutes whereas other adherent populations typically require more than 20-30 minutes incubation. The detached placental stem cells can be harvested following trypsinization and trypsin neutralization, using, e.g., Trypsin Neutralizing Solution (TNS, Cambrex). In one embodiment of isolation of adherent cells, aliquots of, for example, about 5-10×10⁶ cells are placed in each of several T-75 flasks, preferably fibronectin-coated T75 flasks. In such an embodiment, the cells can be cultured with commercially available Mesenchymal Stem Cell Growth Medium (MSCGM) (Cambrex), and placed in a tissue culture incubator (37° C., 5% CO₂). After 10 to 15 days, non-adherent cells are removed from the flasks by washing with PBS. The PBS is then replaced by MSCGM. Flasks are preferably examined daily for the presence of various adherent cell types and in particular, for identification and expansion of clusters of fibroblastoid cells.

The number and type of cells collected from a mammalian placenta can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. For example, using antibodies to CD34, one can determine, using the techniques above, whether a cell comprises a detectable amount of CD34; if so, the cell is CD34+. Likewise, if a cell produces enough OCT-4 RNA to be detectable by RT-PCR, or significantly more OCT-4 RNA than an adult cell, the cell is OCT-4+ Antibodies to cell surface markers (e.g., CD markers such as CD34) and the sequence of stem cell-specific genes, such as OCT-4, are well-known in the art.

Placental cells, particularly cells that have been isolated by Ficoll separation, differential adherence, or a combination of both, may be sorted using a fluorescence activated cell sorter (FACS). Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In one sorting scheme, stem cells from placenta are sorted on the basis of expression of the markers CD34, CD38, CD44, CD45, CD73, CD105, OCT-4 and/or HLA-G. This can be accomplished in connection with procedures to select stem cells on the basis of their adherence properties in culture. For example, an adherence selection stem can be accomplished before or after sorting on the basis of marker expression. In one embodiment, for example, cells are sorted first on the basis of their expression of CD34; CD34⁻ cells are retained, and cells that are CD200⁺ HLA-G⁺, are separated from all other CD34⁻ cells. In another embodiment, cells from placenta are based on their expression of markers CD200 and/or HLA-G; for example, cells displaying either of these markers are isolated for further use. Cells that express, e.g., CD200 and/or HLA-G can, in a specific embodiment, be further sorted based on their expression of CD73 and/or CD105, or epitopes recognized by antibodies SH2, SH3 or SH4, or lack of expression of CD34, CD38 or CD45. For example, in one embodiment, placental cells are sorted by expression, or lack thereof, of CD200, HLA-G, CD73, CD105, CD34, CD38 and CD45, and placental cells that are CD200+, HLA-G+, CD73+, CD105+, CD34⁻, CD38⁻ and CD45⁻ are isolated from other placental cells for further use.

In another embodiment, magnetic beads can be used to separate cells. The cells may be sorted using a magnetic activated cell sorting (MACS) technique, a method for separating particles based on their ability to bind magnetic beads (0.5-100 μm diameter). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one embodiment, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

Placental stem cells can also be characterized and/or sorted based on cell morphology and growth characteristics. For example, placental stem cells can be characterized as having, and/or selected on the basis of, e.g., a fibroblastoid appearance in culture. Placental stem cells can also be characterized as having, and/or be selected, on the basis of their ability to form embryoid-like bodies. In one embodiment, for example, placental cells that are fibroblastoid in shape, express CD73 and CD105, and produce one or more embryoid-like bodies in culture are isolated from other placental cells. In another embodiment, OCT-4⁺ placental cells that produce one or more embryoid-like bodies in culture are isolated from other placental cells.

In another embodiment, placental stem cells can be identified and characterized by a colony forming unit assay. Colony forming unit assays are commonly known in the art, such as MESENCULT™ medium (Stem Cell Technologies, Inc., Vancouver British Columbia)

Placental stem cells can be assessed for viability, proliferation potential, and longevity using standard techniques known in the art, such as trypan blue exclusion assay, fluorescein diacetate uptake assay, propidium iodide uptake assay (to assess viability); and thymidine uptake assay, MTT cell proliferation assay (to assess proliferation). Longevity may be determined by methods well known in the art, such as by determining the maximum number of population doubling in an extended culture.

Placental stem cells can also be separated from other placental cells using other techniques known in the art, e.g., selective growth of desired cells (positive selection), selective destruction of unwanted cells (negative selection); separation based upon differential cell agglutinability in the mixed population as, for example, with soybean agglutinin; freeze-thaw procedures; filtration; conventional and zonal centrifugation; centrifugal elutriation (counter-streaming centrifugation); unit gravity separation; countercurrent distribution; electrophoresis; and the like.

### 5.3. Culturing Stem Cells Using Collagen Biofabric

The present invention provides methods for culturing a stem cell, in particular, culturing an embryonic stem cell or placental stem cell. The methods comprise the step of culturing a stem cell in a culture medium with a collagen biofabric. In one embodiment, the stem cell is exogenous to the collagen biofabric, that is, the stem cell is not from a placenta from which the collagen biofabric is derived.

In some embodiments, the methods comprise culturing a stem cell with a collagen biofabric comprising a plurality of placental stem cells; and culturing said stem cell under conditions appropriate for the survival of the stem cell.

The stem cell can be cultured, e.g., expanded, for a time according to those of skill in the art. In some embodiments, the stem cell is cultured, e.g., expanded, in a culture medium with a collagen biofabric for at least one, two, five, ten, fifteen, twenty or twenty four hours or more. In some embodiments, the stem cell is cultured for at least two, five, seven, ten, fourteen, twenty, twenty five or thirty days or more. In some embodiments, the stem cell is cultured, e.g., expanded, from about two hours to about twenty four hours, from about two hours to about seven days, from about two hours to about fourteen days, from about two hours to about thirty days, from about twenty four hours to about two days, from about twenty four hours to about seven days, from about twenty four hours to about fourteen days, or from about twenty four hours to about thirty days.

The stem cells can be cultured under conditions appropriate for the growth of stem cells well-known to those of skill in the art. The temperature for culturing the stem cells can be, for example, from about 30° C. to about 40° C., from about 30° C. to about 50° C., from about 35° C. to about 40° C., from about 35° C. to about 50° C., from about 35° C. to about 40° C., from about 35° C. to about 45° C., or from about 35° C. to about 50° C. The temperature for culturing the stem cells can be, for example, about 35° C., about 36° C., about 38° C., about 39° C., or about 40° C., preferably about 37° C. The CO₂ level in culturing environment can be, for example, from about 3% CO₂ to about 20% CO₂, from about 5% CO₂ to about 20% CO₂, from about 4% CO₂ to about 10% CO₂, or about 5% CO₂.

General techniques for stem cell culture useful in the practice of the invention are disclosed in, e.g., U.S. Pat. Nos. 6,387,367 and 6,200,806; U.S. Patent Application Publication No. 2006/0057718; see also Teratocarcinomas and Embryonic Stem Cells: A Practical Approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and Uses of Embryonic Stem Cells: Prospects for Application to Human Biology and Gene Therapy (P. D. Rathjen et al., Reprod. Fertil. Dev. 10:31, 1998).

In certain embodiments, the collagen biofabric comprises cells endogenous to a placenta from which the collagen biofabric is derived. Such cells include but are not limited to, placental stem cells, progenitor cells, pluripotent cells and multipotent cells. In some embodiments, the cells are human placental-derived adherent cells.

In certain embodiments, collagen biofabric comprises cells exogenous to a placenta from which the collagen biofabric is derived. Such cells may be, for example, feeder cells, co-cultured with the stem cells of the invention. In some embodiments, the cultured stem cell is a human stem cell and the feeder cells are of human origin. Feeder cells can be any feeder cells known to those of skill in the art, including but not limited to primary mouse embryonic fibroblasts (PMEF), mouse embryonic fibroblast cell line (MEF), murine fetal fibroblasts (MFF), human embryonic fibroblasts (HEF), human fetal muscle cells (HFM), human fetal skin cells (HFS), human adult skin cells, human foreskin fibroblasts (HFF), human adult fallopian tubal epithelial cells (HAFT) or human marrow stromal cells (hMSCs), as described, e.g., in WO 03/02944, WO 03/014313, Park et al., Biol. Reprod., 69:2007-2017, 2003, Amit et al., Biol. Reprod., 68(6):2150-2156, 2003, Hovattal et al., Hum. Reprod., 18 (7): 1404-1409, 2003, Richards et al., Nat Biotechnol., 20(9):933-936, 2002, James et al., Science, 282(6):1145-1147, 1998 and Cheng et al., Stem Cells, 21:131-142, 2003.

In some embodiments, collagen biofabric comprises a combination of cells endogenous and exogenous to a placenta from which the collagen biofabric is derived.

In the present invention, the stem cells cultured with a collagen biofabric are exogenous to the collagen biofabric. In some embodiments, the collagen biofabric is processed to remove all endogenous cells to allow exogenous stem cells to be cultured. Methods for removal of the endogenous cells are well-known in the art. For example, endogenous cells can be removed using a mild detergent, e.g., deoxycholic acid. In another embodiment, endogenous cells are killed prior to culturing a stem cell. Methods of killing cells are well-known in the art. For example, the collagen biofabric can be irradiated with electromagnetic, UV, X-ray, gamma- or beta-radiation to eradicate all remaining viable endogenous cells. In one embodiment, sub-lethal exposure to radiations e.g., 500 to 1500 cGy can be used to preserve the placenta but eradicate undesired cells. Chapter 5 “Biophysical and Biological Effects of Ionizing Radiation” from the United States Department of Defense, for example, provides international standards on lethal v. non-lethal ionizing radiation.

The stem cells may be plated onto the collagen biofabric in a suitable distribution manner and in the presence of a culture medium that promotes cell survival and growth. The stem cells can be plated on the collagen biofabric at any time and in any manner according to the judgment of those of skill in the art. For example, the collagen biofabric may be deposited to a stem cell culture at the time of passaging the cells or as part of a regular feeding. Alternatively, the stem cells may be plated onto the collagen biofabric directly after isolation.

The number of stem or progenitor cells plated onto the surface of the collagen biofabric can vary, but may be at least 1×10³, 3×10³, 1×10⁴, 3×10⁴, 1×10⁵, 3×10⁵, 1×10⁶, 3×10⁶, 1×10 stem cells; or may be no more than 1×10³, 3×10³, 1×10⁴, 3×10⁴, 1×10⁵, 3×10⁵, 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹² stem or progenitor cells.

In another embodiment of any of the culturing embodiments herein, the stem cells are cultured on a collagen-based biomaterial derived from umbilical cord, e.g., from the umbilical cord membrane. In a preferred embodiment, the umbilical cord-derived biomaterial is decellularized and processed substantially as disclosed herein for the preparation of collagen biofabric. Preferably the stem cells are cultured on substantially flat sheets or pieces of the umbilical cord biomaterial.

5.3.1. Culture Medium

Once isolated, the stems cells are cultured in a culture medium with a collagen biofabric. The culture medium can be any culture medium suitable for culturing stem cells, for example, culture medium suitable for culturing stem cells in a feeder cell free condition. Such culture medium includes but is not limited to those described in U.S. Pat. No. 6,800,480, U.S. Application Publication No. 2005/0153445. In a specific embodiment, culture medium that can be used in the present invention comprises about 500 mL distilled water; 60 mL DMEM (Gibco-BRL); about 40 mL MCDB201 (Sigma) dissolved in water, pH 7.2; about 2 mL FCS (Hyclone); about 1 mL 100×ITS (insulin transferrin selenium; Sigma); pen&strep; about 10 ng/mL LA; bovine serum albumin; about 50 nM dexamethasone (Sigma); about 10 ng/ml PDGF (platelet-derived growth factor; and about 10 ng/mL EGF (epidermal growth factor)

The media utilized may or may not comprise serum, although those of skill in the art recognizes that it may be advantageous to use serum-free media so that the cells are not exposed to serum-borne pathogens.

Those of skill in the art would recognize that the culture media may be supplemented with one or more expansion factors to facilitate culturing or expansion, depending on the tissue from which the stem cells originally derive or to the tissue for which they will differentiate into. For example, for embryonic stem cells, expansion factors ex vivo may include one or more of the following: FGFβ, Wnt-3a, collagen, fibronectin, and laminin. For mesenchymal stem cells, for example, expansion factors ex vivo may include one or more FGFβ, EGF, PDGF, and fibronectin. For hematopoietic stem cells, expansion factors ex vivo may include one or more of IL-3, IL-6, SCF, Flt-3/Flk-2, Tpo, Shh, Wnt-3a, and Kirre. For neural stem cells, ex vivo expansion factors may include FGFβ, EGF, fibronectin, and cystatin C.

In some embodiments, conditioned medium is used with collagen biofabric for culturing stem cells. Conditioned medium as used herein refers to medium in which feeder cells have been cultivated already for a period of time.

Those of skill in the art will understand that different culture media can be used depending upon the purpose of culturing (culturing, expanding or differentiating a stem cell), the source from which the stem cells are derived, the type of cells into which the stem cells can be induced to differentiate, collagen biofabric used in culture and the presence or absence of cells other than the stem cells.

### 5.4. Expanding Stem Cells Using Collagen Biofabric

The present invention provides methods for expanding a stem cell or population of stem cells comprising culturing a stem cell or population of stem cells in a culture medium with a collagen biofabric under conditions that allow said stem cell or population of stem cells to expand.

The stem cell or population of stem cells can be cultured under appropriate conditions and for a time according to those of skill in the art. In some embodiments, the stem cell is cultured in a culture medium with a collagen biofabric for twenty four hours or more. In some embodiments, the stem cell is cultured for two days or more. In some embodiments, the stem cell is cultured for seven days or more. In some embodiments, the stem cell is cultured for ten days or more. In some embodiments, the stem cell is cultured for fourteen days or more. In some embodiments, the stem cell is cultured for thirty days or more.

In some embodiments, a single stem cell, or about, or at least, or at most 10, 20, 50, 100, 200, 500, 1×10³, 5×10³, 1×10⁴ or 5×10⁴ stem cells are expanded in a culture medium with a collagen biofabric. In other embodiments, stem cells are cultured and expanded in accordance with the method of the invention and the number of stem cells is increased 2, 5, 10, 20, 50, 100, 200, 500, 1×10³, 5×10³, 1×10⁴ or 5×10⁴ times in comparison to the number of stem cells originally cultured. In other embodiments, the number of stem cells in culture is increased to about, or at least, 1×10⁶, 5×10⁶, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, or 1×10¹² stem cells; or may be no more than 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, or 1×10¹² stem cells.

### 5.5. Differentiating Stem Cells Using Collagen Biofabric

The invention provides methods for differentiating a stem cell, comprising culturing a stem cell in a culture medium with a collagen biofabric for a time sufficient for differentiation of the stem cell. The invention encompasses methods of the differentiating a stem cell into a specific cell lineage, including, but not limited to, a mesenchymal, hematopoietic, adipogenic, hepatogenic, neurogenic, gliogenic, chondrogenic, vasogenic, myogenic, pancreagenic, chondrogenic, or osteogenic lineage.

Those of skill in the art would understand the time sufficient for differentiation of a stem cell may vary depending on the type of the stem cell cultured and the cell type to which the stem cell is differentiated. In some embodiments, the stem cell is cultured in a culture medium with a collagen biofabric for at least, about, or at most one, two, five, ten, fifteen, twenty or twenty four hours or more. In some embodiments, the stem cell is cultured at least, about, or at most two, five, seven, ten, fourteen, twenty, twenty five or thirty days or more. In some embodiments, the stem cell is cultured from about two hours to about twenty four hours, from about two hours to about seven days, from about two hours to about fourteen days, from about two hours to about thirty days, from about twenty four hours to about two days, from about twenty four hours to about seven days, from about twenty four hours to about fourteen days, or from about twenty four hours to about thirty days.

In certain embodiments, the methods further comprise contacting the stem cell with one or more agents that facilitate the desired differentiation. For example, the agent may induce or facilitate a change in phenotype, promote growth of cells with a particular phenotype or slow the growth of others, or act in concert with other agents through an unknown mechanism. Such agents can be small molecules or cytokines, such as those disclosed in U.S. Application Publication Nos. 2003/0235909, 2004/0028660 (small molecules), U.S. Pat. No. 6,335,195 (hematopoietic and mesenchymal stem cell in the presence of angiotensinogen and angiotensin), U.S. Pat. No. 6,022,743 (pancreatic parenchymal cells-three dimensional culture), U.S. Pat. No. 6,613,568 (hematopoietic lineage).

Stem cells can be differentiated in any culture media or conditions suitable for the differentiation of the stem cell, such as described in, e.g., U.S. Application Publication Nos. 2005/015344 and 2005/0158855 (general), U.S. Pat. Nos. 6,833,269 and 6,887,706 and U.S. Application Publication No. 2005/0095706 (neural cells), U.S. Application Publication No. 2005/0170502 (hepatic lineage), Kehat, 2003, Methods in Enzymology 365:465-473, U.S. Application Publication Nos. 2005/0191744 and 2005/0214939 (cardiac cells), and Assady et al., 2001, Diabetes, 50:1691-97 (pancreatic cells).

Assessment of the differentiation state of stem cells obtained according to the methods of the invention may be identified by the presence or absence of certain cell surface markers. Placental stem cells, for example, may be identified by the markers OCT-4 and ABC-p, or the equivalents thereof in different mammalian species. Placental stem cells can also be identified by presence of the markers CD73 or CD105, and/or the absence of the markers CD34, CD38, or CD45, or the equivalents thereof in different mammalian species. In certain embodiments, the placental stem cells are positive for SSEA3 and/or SSEA4. In certain other embodiments, the placental stem cells are negative for SSEA3 and/or SSEA4. The presence or absence of such cell surface markers can be routinely determined according to methods well known in the art, e.g., by flow cytometry. For example, to determine the presence of CD34 or CD38, cells may be washed in PBS and then double-stained with anti-CD34 phycoerythrin and anti-CD38 fluorescein isothiocyanate (Becton Dickinson, Mountain View, Calif.).

In another embodiment, differentiated stem cells are identified and characterized by a colony forming unit assay, which is commonly known in the art, such as MESENCULT™ medium (Stem Cell Technologies, Inc., Vancouver British Columbia).

Determination that a stem cell has differentiated into a particular cell type can be accomplished by methods well-known in the art, e.g., measuring changes in morphology and cell surface markers using techniques such as flow cytometry or immunocytochemistry (e.g., staining cells with tissue-specific or cell-marker specific antibodies), by examination of the morphology of cells using light or confocal microscopy, or by measuring changes in gene expression using techniques well known in the art, such as PCR and gene-expression profiling.

In certain embodiments, differentiated cells may be identified by characterizing differentially expressed genes, for example, by comparing the level of expression of a plurality of genes from an undifferentiated stem or progenitor cell of interest to the level of expression of said plurality of genes in a differentiated cell derived from that type of progenitor cell. For example, nucleic acid amplification methods such as polymerase chain reaction (PCR) or transcription-based amplification methods (e.g., in vitro transcription (IVT)) may be used to profile gene expression in different populations of cells, e.g., by use of a polynucleotide microarray. Such methods to profile differential gene expression are well known in the art. See, e.g., Wieland et al., 1990, Proc. Natl. Acad. Sci. USA 87: 2720-2724; Lisitsyn et al., 1993, Science 259: 946-951; Lisitsyn et al., 1995, Meth. Enzymol. 254:291-304; U.S. Pat. No. 5,436,142; U.S. Pat. No. 5,501,964; Lisitsyn et al., 1994, Nature Genetics 6:57-63; Hubank and Schatz, 1994, Nucleic Acids Res. 22: 5640-5648; Zeng et al., 1994, Nucleic Acids Research 22: 4381-4385; U.S. Pat. No. 5,525,471; Linsley et al., U.S. Pat. No. 6,271,002; Van Gelder et al., U.S. Pat. No. 5,716,785; Stoflet et al., 1988, Science 239:491-494; Sarkar and Sommer, 1989, Science 244:331-334; Mullis et al., U.S. Pat. No. 4,683,195; Malek et al., U.S. Pat. No. 5,130,238; Kacian and Fultz, U.S. Pat. No. 5,399,491; Burg et al., U.S. Pat. No. 5,437,990; van Gelder et al., 1990, Proc. Natl. Acad. Sci. USA 87:1663; Lockhart et al., 1996, Nature Biotechnol. 14:1675; Shannon, U.S. Pat. No. 6,132,997; Lindemann et al., U.S. Pat. No. 6,235,503.

5.5.1. Differentiation into a Neural Cell

In one aspect, the present invention encompasses a method for the differentiation of a stem cell into a neural cell comprising culturing the stem cell on collagen biofabric under conditions that promote differentiation of the stem cell into a neural cell. In certain embodiments, the method comprises the step of contacting the stem cell with one or more agents that facilitate the differentiation of a stem cell into a neural cell. Exemplary agents include, but are not limited to, betamercaptoethanol (Woodbury et al., J. Neurosci. Res., 61:364-370) or butylated hydroxyanisole. In some embodiments, the collagen biofabric comprises the one or more agents. Any culture medium suitable for neural differentiation known in the art may be used in the cell culture. For example, differentiation can be induced by culturing a stem cell in DMEM medium containing 2% DMSO and 200 μM butylated hydroxyanisole until differentiation is observed.

Assessment and determination that a stem cell has differentiated into a neural cell type may be accomplished by any methods known in the art. For example, RT/PCR may be used to assess the expression of e.g., nerve growth factor receptor and neurofilament heavy chain genes. In some embodiments, the neural cell exhibits production of nerve growth factor receptor; expression of a gene encoding nerve growth factor; production of neurofilament heavy chain; or expression of a gene encoding neurofilament heavy chain.

5.5.2. Differentiation into an Adipocyte Cell

In another aspect, the present invention encompasses methods of differentiating a stem cell into an adipocyte comprising culturing the stem cell on collagen biofabric under conditions that promote differentiation of the stem cell into an adipocyte cell. In some embodiments, the adipocyte cell exhibits production of intracytoplasmic lipid vesicles detectable by a lipophilic stain; expression of a gene encoding lipase; or production of lipase. In certain embodiments, differentiation comprises contacting the stem cell with collagen biofabric and one or more agents that facilitate the differentiation of a stem cell into an adipocyte. Exemplary agents are dexamethasone, indomethacin, insulin, and 3-isobutyl-1-methylxanthine. In some embodiments, the collagen biofabric comprises the one or more agents.

Any culture medium suitable for adipocyte differentiation known in the art may be used in the cell culture. For example, Adipogenesis Maintenance Medium (Bio Whittaker) containing 1 μM dexamethasone, 0.2 mM indomethacin, 0.01 mg/ml insulin, 0.5 mM IBMX, DMEM-high glucose, FBS, and antibiotics may be used to induce the differentiation.

Determination that a stem cell has differentiated into an adipocyte cell type may be accomplished by methods known in the art. For instance, adipogenesis may be assessed by the development of multiple intracytoplasmic lipid vesicles that can be easily observed using the lipophilic stain oil red O. Differentiation can also be established by detecting expression of lipase and fatty acid binding protein genes using, e.g., RT-PCR.

5.5.3. Differentiation into a Chondrocyte Cell

In another aspect, the present invention encompasses methods of differentiating a stem cell into a chondrocyte comprising culturing the stem cell on collagen biofabric under conditions that promote differentiation of the stem cell into a chondrocyte. In some embodiments, the chondrocyte exhibits cell morphology characteristic of a chondrocyte; production of collagen 2; expression of a gene encoding collagen 2, production of collagen 9; or expression of a gene encoding collagen 9. In certain embodiments, differentiation comprises contacting the stem cell with collagen biofabric alone and one or more agents that facilitate the differentiation of a stem cell into a chondrocyte cell. Exemplary agents are transforming growth factor-beta-3. In some embodiments, the collagen biofabric comprises the one or more agents.

Any culture medium suitable for chondrocyte differentiation known in the art may be used in the cell culture. For example, Complete Chondrogenesis Medium (Bio Whittaker) containing 0.01 μg/ml TGF-beta-3 may be used to induce the differentiation.

Determination that a stem cell has differentiated into a chondrocyte cell type may be accomplished by methods known in the art. For instance, chondrogenesis may be established by, e.g., observation of production of esoinophilic ground substance, development of chondrocyte cell morphology, and/or detection of collagen 2 and collagen 9 gene expression using, e.g., RT-PCR.

5.5.4. Differentiation into an Osteocyte

In another aspect, the present invention encompasses methods of differentiating a stem cell into an osteocyte cell comprising culturing the stem cell on collagen biofabric under conditions that promote differentiation of the stem cell into an osteocyte. In some embodiments, the osteocyte cell exhibits calcium levels characteristic of an osteocyte; production of alkaline phosphatase; expression of a gene encoding alkaline phosphatase; production of osteopontin; or expression of a gene encoding osteopontin. In certain embodiments, differentiation comprises contacting the stem cell with collagen biofabric and one or more agents that facilitate the differentiation of a stem cell into an osteocyte known in the art. Exemplary agents are dexamethasone, ascorbic acid-2-phosphate, and glycerophosphate. In some embodiments, the collagen biofabric comprises the one or more agents.

Any culture medium suitable for osteocyte differentiation known in the art may be used in the cell culture. For example, Osteogenic Induction Medium (Bio Whittaker) containing 0.1 μM dexamethasone, 0.05 mM ascorbic acid-2-phosphate, 10 mM beta glycerophosphate may be used to induce the differentiation.

Determination that a stem cell has differentiated into an osteocyte may be accomplished by methods known in the art. For instance, differentiation can be evidenced using a calcium-specific stain and detection of alkaline phosphatase and/or osteopontin gene expression using, e.g., RT-PCR.

5.5.5. Differentiation into a Hepatocyte Cell

In another aspect, the present invention encompasses methods of differentiating a stem cell into a hepatocyte cell comprising culturing the stem cell on collagen biofabric under conditions that promote differentiation of the stem cell into a hepatocyte. In some embodiments, the hepatocyte cell exhibits expression of a hepatocyte-specific gene or production of a hepatocyte-specific protein. Such genes and proteins are known in the art and can be albumin, pre-albumin, glucose-6-phosphatase, al-antitrypsin etc., as described in U.S. Application Publication No. 2005/0170502.

Differentiation can comprise contacting the stem cell with collagen biofabric and one or more agents that facilitate the differentiation of a stem cell into a hepatocyte. For example, hepatocyte growth factor and/or epidermal growth factor. In some embodiments, the collagen biofabric comprises the one or more agents. Any culture medium suitable for hepatocyte differentiation known in the art may be used in the cell culture. For example, DMEM medium with 20% CBS supplemented with hepatocyte growth factor, 20 ng/ml; and epidermal growth factor, 100 ng/ml may be used to induce the differentiation. KnockOut Serum Replacement may be used in lieu of FBS.

Differentiation into a hepatocyte may be evidenced by detection of production of albumin, pre-albumin, glucose-6-phosphatase, al-antitrypsin, or expression of a gene encoding the same.

5.5.6. Differentiation into a Pancreatic Cell

In another aspect, the present invention encompasses methods of differentiating a stem cell into a pancreatic cell comprising culturing the stem cell on collagen biofabric under conditions that promote differentiation of the stem cell into a pancreatic cell. In some embodiments, the pancreatic cell exhibits production of insulin or expression of a gene encoding insulin.

Differentiation can comprise contacting the stem cell with collagen biofabric and one or more agents that facilitate the differentiation of a stem cell into a pancreatic cell known in the art. Exemplary agents are basic fibroblast growth factor, transforming growth factor beta-1, and medium conditioned by nestin-positive neuronal cells. In some embodiments, the collagen biofabric comprises the one or more agents. Any culture medium suitable for pancreatic cell differentiation known in the art may be used in the cell culture. For example, conditioned media from nestin-positive neuronal cell cultures mixed with DMEM medium may be used.

Determination that a stem cell has differentiated into a pancreatic cell may be accomplished by methods known in the art. For example, differentiation can be evidenced by detection of production of insulin, or of insulin gene expression using, e.g., RT-PCR.

5.5.7. Differentiation into a Cardiac Cell

In another aspect, the present invention encompasses methods of differentiating a stem cell into a cardiac cell comprising culturing the stem cell on collagen biofabric under conditions that promote differentiation of the stem cell into a cardiac cell. In some embodiments, the cardiac cell exhibits beating; production of cardiac actin; or expression of a gene encoding cardiac actin.

Differentiation can comprise contacting the stem cell with one or more agents that facilitate the differentiation of a stem cell into a cardiac cell. Exemplary agents include retinoic acid, basic fibroblast growth factor, transforming growth factor or cardiotropin. In some embodiments, the collagen biofabric comprises the one or more agents.

Any culture medium suitable for cardiac cell differentiation known in the art may be used in the cell culture. For example, DMEM medium with 20% CBS, supplemented with retinoic acid, 1 μM; basic fibroblast growth factor, 10 ng/ml; and transforming growth factor beta-1, 2 ng/ml; and epidermal growth factor, 100 ng/ml may be used in the culture. KnockOut Serum Replacement (Invitrogen, Carlsbad, Calif.) may be used in lieu of CBS. Alternatively, DMEM medium with 20% CBS supplemented with 50 ng/ml Cardiotropin-1 may be used. Besides, stem cells may be maintained in protein-free media for 5-7 days, then stimulated with human myocardium extract (escalating dose analysis). Myocardium extract is produced by homogenizing 1 gm human myocardium in 1% HEPES buffer supplemented with 1% cord blood serum. The suspension is incubated for 60 minutes, then centrifuged and the supernatant collected.

Determination that a stem cell has differentiated into a cardiac cell type may be accomplished by methods known in the art. For example, differentiation is evidenced by, e.g., beating, production of cardiac actin, or expression of a gene encoding cardiac actin.

### 5.6. Collagen Biofabric

The present invention provides methods of culturing, expanding or differentiating a stem cell using a collagen biofabric. While not intending to be limited by any theory, it is contemplated that the collagen biofabric provides substratum for cell attachment and appropriate growth factors for stem cell growth in culture.

The collagen biofabric can be used in dry or native form (that is, as dissected from a placenta), and/or in decellularized or non-decellularized form.

In some embodiments, the collagen biofabric comprises cells endogenous to a placenta from which the collagen biofabric is derived. In other embodiments, the collagen biofabric comprises cells exogenous to a placenta from which the collagen biofabric is derived. In some embodiments, the collagen biofabric comprises both cells exogenous and cells endogenous to a placenta from which the collagen biofabric is derived.

### 5.6.1. Description

The collagen biofabric used in the present invention may be derived from the amniotic membrane, chorionic membrane, or both of any mammal, for example, equine, bovine, porcine or catarrhine sources, but is most preferably derived from human placenta. In a preferred embodiment, the collagen biofabric is substantially dry, i.e., is 20% or less water by weight. In another preferred embodiment, the collagen biofabric has not been protease-treated. In another preferred embodiment, the collagen biofabric contains no collagen and other structural proteins that have been artificially crosslinked, e.g., chemically crosslinked, that is, the preferred collagen biofabric is not fixed. A preferred collagen biofabric is the dried, non-fixed, non-protease-treated amniotic membrane material described in Hariri, U.S. Application Publication U.S. 2004/0048796, which is hereby incorporated in its entirety, and that is produced by the methods described therein, and herein (see Examples 1, 2). However, the methods of the present invention can utilize any placental collagen material made by any procedure.

In a preferred embodiment, the collagen biofabric is translucent. In other embodiments, the collagen biofabric is opaque, or is colored or dyed, e.g., permanently colored or dyed, using a medically-acceptable dyeing or coloring agent; such an agent may be adsorbed onto the collagen biofabric, or the collagen biofabric may be impregnated or coated with such an agent. In this embodiment, any known non-toxic, non-irritating coloring agent or dye may be used.

When the collagen biofabric is substantially dry, it is about 0.1 g/cm² to about 0.6 g/cm². In a specific embodiment, a single layer of the collagen biofabric is at least 2 microns in thickness. In another specific embodiment, a single layer of the collagen biofabric used to repair a tympanic membrane is approximately 10-40 microns in thickness, but may be approximately 2-150, 2-100 microns, 5-75 microns or 7-60 microns in thickness in the dry state.

In one embodiment, the collagen biofabric is principally composed of collagen (types I, III and IV; about 90% of the matrix of the biofabric), fibrin, fibronectin, elastin, and further contains glycosaminoglycans and proteoglycans. In other embodiments, non-structural components of the biofabric may include, for example, growth factors, e.g., platelet-derived growth factors (PDGFs), vascular-endothelial growth factor (VEGF), fibroblast growth factor (FGF) and transforming growth factor-β1. The composition of the collagen biofabric is thus ideally suited to encourage the migration of fibroblasts and macrophages, and thus the promotion of wound healing.

The collagen biofabric may be used in a single-layered format, for example, as a single-layer sheet or an un-laminated membrane. Alternatively, the collagen biofabric may be used in a double-layer or multiple-layer format, e.g., the collagen biofabric may be laminated. Lamination can provide greater stiffness and durability during the healing process. The collagen biofabric may be, for example, laminated as described below.

The collagen biofabric may further comprise collagen from a non-placenta source. For example, one or more layers of collagen biofabric may be coated or impregnated with, or layered with, purified extracted collagen. Such collagen may be obtained, for example, from commercial sources, or may be produced according to known methods, such as those disclosed in U.S. Pat. Nos. 4,420,339, 5,814,328, and 5,436,135.

The collagen biofabric may comprise cells endogenous to a placenta from which the collagen biofabric is derived. The collagen biofabric may also comprise cells exogenous to a placenta from which the collagen biofabric is derived. In some embodiments, the collagen biofabric may comprise both cells exogenous and cells endogenous to a placenta from which the collagen biofabric is derived.

The collagen biofabric may comprise one or more compounds or substances that are not present in the placental material from which the collagen biofabric is derived. For example, the collagen biofabric may be impregnated with a bioactive compound. Such bioactive compounds include, but are not limited to, small organic molecules (e.g., drugs), antibiotics (such as Clindamycin, Minocycline, Doxycycline, Gentamycin), hormones, growth factors, anti-tumor agents, anti-fungal agents, anti-viral agents, pain medications, anti-histamines, anti-inflammatory agents, anti-infectives including but not limited to silver (such as silver salts, including but not limited to silver nitrate and silver sulfadiazine), elemental silver, antibiotics, bactericidal enzymes (such as lysozyme), wound healing agents (such as cytokines including but not limited to PDGF, TGF; thymosin), hyaluronic acid as a wound healing agent, wound sealants (such as fibrin with or without thrombin), cellular attractant and scaffolding reagents (such as added fibronectin) and the like. In a specific example, the collagen biofabric may be impregnated with at least one growth factor, for example, fibroblast growth factor, epithelial growth factor, etc. The biofabric may also be impregnated with small organic molecules such as specific inhibitors of particular biochemical processes e.g., membrane receptor inhibitors, kinase inhibitors, growth inhibitors, anticancer drugs, antibiotics, etc. Impregnating the collagen biofabric with a bioactive compound may be accomplished, e.g., by immersing the collagen biofabric in a solution of the bioactive compound of the desired concentration for a time sufficient to allow the collagen biofabric to absorb and to equilibrate with the solution; by spraying the solution onto the biofabric; by wetting the biofabric with the solution, etc.

In other embodiments, the collagen biofabric may be combined with a hydrogel. Any hydrogel composition known to one skilled in the art is encompassed within the invention, e.g., any of the hydrogel compositions disclosed in the following reviews: Graham, 1998, Med. Device Technol. 9(1): 18-22; Peppas et al., 2000, Eur. J. Pharm. Biopharm. 50(1): 27-46; Nguyen et al., 2002, Biomaterials, 23(22): 4307-14; Henincl et al., 2002, Adv. Drug Deliv. Rev 54(1): 13-36; Skelhorne et al., 2002, Med. Device. Technol. 13(9): 19-23; Schmedlen et al., 2002, Biomaterials 23: 4325-32; all of which are incorporated herein by reference in their entirety. In a specific embodiment, the hydrogel composition is applied on the collagen biofabric, i.e., disposed on the surface of the collagen biofabric. The hydrogel composition for example, may be sprayed onto the collagen biofabric or coated onto the surface of the collagen biofabric, or the biofabric may be soaked, bathed or saturated with the hydrogel composition. In another specific embodiment, the hydrogel is sandwiched between two or more layers of collagen biofabric. In an even more specific embodiment, the hydrogel is sandwiched between two or more layers of collagen biofabric, wherein the edges of the two layers of biofabric are sealed so as to substantially or completely contain the hydrogel.

The hydrogels useful in the methods and compositions of the invention can be made from any water-interactive, or water soluble polymer known in the art, including but not limited to, polyvinylalcohol (PVA), polyhydroxyethyl methacrylate, polyethylene glycol, polyvinyl pyrrolidone, hyaluronic acid, alginate, collagen, gelatin, dextran or derivatives and analogs thereof.

In a specific embodiment, the collagen biofabric comprises hyaluronic acid. The hyaluronic acid can be, e.g., applied or added to the collagen biofabric as a solution, e.g., a 10 mg/mL solution in, e.g., water or a physiologically-acceptable buffer or culture medium. The hyaluronic acid is preferably sufficiently crosslinked to reduce or prevent solubility of the hyaluronic acid in a liquid environment. In preferred embodiments, the hyaluronic acid is crosslinked to the collagen biofabric. The crosslinking agent, for crosslinking hyaluronic acid, or for crosslinking the hyaluronic acid to the collagen biofabric, can be any crosslinking agent, but can be, for example, 1,4-butanediol diglycidyl ether (BDDE), 1-ethyl-3-(3-dimethylaminopropyl)carbodiimide hydrochloride (EDCI), divinyl sulfone, epichlorohydrin, glutaraldehyde, dicyclohexylcarbodiimide (DCC), or the like. The combination of collagen biofabric and hyaluronic acid can optionally be dried, e.g., air dried, lyophilized, or the like. In certain embodiments, the collagen biofabric is placed into a frame or holder that, e.g., holds the collagen biofabric at the edges, prior to and during addition of the hyaluronic acid.

The invention also provides a method of manufacturing a collagen biofabric comprising hyaluronic acid, e.g., to be used for the culture of a stem cell or population of stem cells, e.g., adherent, CD34⁻ placental stem cells, comprising contacting at least a portion of a collagen biofabric with a hyaluronic acid solution, crosslinking the hyaluronic acid to the collagen biofabric, and drying the resulting collagen biofabric. In one embodiment of the method, the collagen biofabric is substantially dry, e.g., comprises 20% or less water, at the time of contacting with the hyaluronic acid solution. In another specific embodiment of the method, the collagen biofabric is decellularized prior to contacting with the hyaluronic acid solution. In another embodiment, the collagen biofabric is sheet-like in appearance. In another specific embodiment of the method, the collagen biofabric is not decellularized prior to contacting with the hyaluronic acid solution. Preferably, the collagen biofabric, at the time of contacting with the hyaluronic acid solution, is held on one or more sides, e.g., in a frame, to reduce the amount of, or prevent, curling of the collagen biofabric during contacting. In a particular embodiment, the collagen biofabric is a square or rectangular sheet, and is held in a four-sided frame that contacts all four edges of the collagen biofabric during contacting with a hyaluronic acid solution.

For the composition and method embodiments above, the hyaluronic acid solution can be any hyaluronic acid solution that allows for even distribution of the hyaluronic acid on the surface of the portion of the collagen biofabric contacted. For example, the hyaluronic acid solution can comprise at least, about, or at most 0.1, 0.2, 0.3, 0.4, 0.5, 0.6, 0.7, 0.8, 0.9, 1, 2, 3, 4, 5, 6, 7, 8, 9, 10, 12, 14, 16, 18, 20, 25, 30, 35, 40, 45, 50, 55, 60, 65, 70, 75, 80, 85, 90, 95 or 100 mg hyaluronic acid per milliliter of solution.

In some embodiments, the collagen biofabric comprises one or more bioactive compounds and is combined with a hydrogel. For example, the collagen biofabric can be impregnated with one or more bioactive compounds prior to being combined with a hydrogel. In other embodiments, the hydrogel composition is further impregnated with one or more bioactive compounds prior to, or after, being combined with a collagen biofabric of the invention, for example, the bioactive compounds described in section below.

### 5.6.2. Bioactive Compounds

The collagen biofabric used in the methods of the invention may comprise (e.g., be impregnated with or coated with) one or more bioactive compounds. As used herein, the term “bioactive compound” means any compound or molecule that causes a measurable effect on one or more biological systems in vitro or in vivo. Examples of bioactive compounds include, without limitation, small organic molecules (e.g., drugs), antibiotics, antiviral agents, antimicrobial agents, anti-inflammatory agents, antiproliferative agents, cytokines, enzyme or protein inhibitors, antihistamines, and the like. In various embodiments, the collagen biofabric may be coated or impregnated with antibiotics (such as Clindamycin, Minocycline, Doxycycline, Gentamycin), hormones, growth factors, anti-tumor agents, anti-fungal agents, anti-viral agents, pain medications (including XYLOCAINE®, Lidocaine, Procaine, Novocaine, etc.), antihistamines (e.g., diphenhydramine, BENADRYL®, etc.), anti-inflammatory agents, anti-infectives including but not limited to silver (such as silver salts, including but not limited to silver nitrate and silver sulfadiazine), elemental silver, antibiotics, bactericidal enzymes (such as lysozome), wound healing agents (such as cytokines including but not limited to PDGF (e.g., REGRANEX®), TGF; thymosin), hyaluronic acid as a wound healing agent, wound sealants (such as fibrin with or without thrombin), cellular attractant and scaffolding reagents (such as fibronectin), and the like, or combinations of any of the foregoing, or of the foregoing and other compounds not listed. Such impregnation or coating may be accomplished by any means known in the art, and a portion or the whole of the collagen biofabric may be so coated or impregnated.

The collagen biofabric, or composites comprising collagen biofabric, may comprise any of the compounds listed herein, without limitation, individually or in any combination. Any of the biologically active compounds listed herein, and others useful in the context of the sclera or eye, may be formulated by known methods for immediate release or extended release. Additionally, the collagen biofabric may comprise two or more biologically active compounds in different manners; e.g., the biofabric may be impregnated with one biologically active compound and coated with another. In another embodiment, the collagen biofabric comprises one biologically active compound formulated for extended release, and a second biologically active compound formulated for immediate release.

The collagen biofabric may be impregnated or coated with a physiologically-available form of one or more nutrients required for wound healing. Preferably, the nutrient is formulated for extended release.

The collagen biofabric, or composite comprising collagen biofabric, may comprise an antibiotic. In certain embodiments, the antibiotic is a macrolide (e.g., tobramycin (TOBI)), a cephalosporin (e.g., cephalexin (KEFLEX®)), cephradine (VELOSEF®)), cefuroxime (CEFTIN®, cefprozil (CEFZI®), cefaclor (CECLOR®), cefixime (SUPRAX® or cefadroxil (DURICEF®), a clarithromycin (e.g., clarithromycin (Biaxin)), an erythromycin (e.g., erythromycin (EMYCIN®)), a penicillin (e.g., penicillin V (V-CILLINK® or PEN VEEK®)) or a quinolone (e.g., ofloxacin (FLOXIN®), ciprofloxacin (CIPRO®) ornorfloxacin (NOROXIN®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

In certain embodiments, the collagen biofabric may be coated or impregnated with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

In certain other embodiments, the collagen biofabric, or a composite comprising collagen biofabric, is coated or impregnated with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, methotrexate, colchicine, allopurinol, probenecid, sulfinpyrazone and benzbromarone.

In certain embodiments, the collagen biofabric, or a composite comprising collagen biofabric, is coated or impregnated with an antiviral agent. Useful antiviral agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons.

The collagen biofabric, or a composite comprising collagen biofabric, may also be coated or impregnated with a cytokine receptor modulator. Examples of cytokine receptor modulators include, but are not limited to, soluble cytokine receptors (e.g., the extracellular domain of a TNF-α receptor or a fragment thereof, the extracellular domain of an IL-10 receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e. g., anti-IFN antibodies, anti-TNF-α antibodies, anti-IL-10 antibodies, anti-IL-6 antibodies, anti-IL-8 antibodies (e.g., ABX-IL-8 (Abgenix)), and anti-IL-12 antibodies). In a specific embodiment, a cytokine receptor modulator is IL-4, IL-10, or a fragment thereof. In another embodiment, a cytokine receptor modulator is an anti-IL-1 antibody, anti-IL-6 antibody, anti-IL-12 receptor antibody, or anti-TNF-α antibody. In another embodiment, a cytokine receptor modulator is the extracellular domain of a TNF-α receptor or a fragment thereof. In certain embodiments, a cytokine receptor modulator is not a TNF-α antagonist.

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

The collagen biofabric, or a composite comprising collagen biofabric, may also be coated or impregnated with a cytokine. Examples of cytokines include, but are not limited to, colony stimulating factor 1 (CSF-1), interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), insulin-like growth factor 1 (IGF-1), platelet derived growth factor (PDGF), erythropoietin (Epo), epidermal growth factor (EGF), fibroblast growth factor (FGF) (basic or acidic), granulocyte macrophage stimulating factor (GM-CSF), granulocyte colony stimulating factor (G-CSF), heparin binding epidermal growth factor (HEGF), macrophage colony stimulating factor (M-CSF), prolactin, and interferon (IFN), e.g., IFN-alpha, and IFN-gamma), transforming growth factor alpha (TGF-α), TGFβ1, TGFβ2, tumor necrosis factor alpha (TNF-α), vascular endothelial growth factor (VEGF), hepatocyte growth factor (HGF), etc.

The collagen biofabric may also be coated or impregnated with a hormone. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins. Examples of β-interferons include, but are not limited to, interferon β1-a and interferon β1-b.

The collagen biofabric, or composite comprising collagen biofabric, may also be coated or impregnated with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The collagen biofabric, or a composite comprising collagen biofabric, may also be coated or impregnated with an immunomodulatory agent, including but not limited to methothrexate, leflunomide, cyclophosphamide, cyclosporine A, macrolide antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators. peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators. Examples of T cell receptor modulators include, but are not limited to, anti-T cell receptor antibodies (e.g., anti-CD4 antibodies (e.g., cM-T412 (Boehringer), IDEC-CE9.Is (IDEC and SKB), mAb 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131(IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114) (IDEC))) and CTLA4-immunoglobulin. In a specific embodiment, a T cell receptor modulator is a CD2 antagonist. In other embodiments, a T cell receptor modulator is not a CD2 antagonist. In another specific embodiment, a T cell receptor modulator is a CD2 binding molecule, preferably MEDI-507. In other embodiments, a T cell receptor modulator is not a CD2 binding molecule.

The collagen biofabric, or composite comprising collagen biofabric, may also be coated or impregnated with a class of immunomodulatory compounds known as IMIDs®. As used herein and unless otherwise indicated, the term IMID® and IMIDs® (Celgene Corporation) encompasses small organic molecules that markedly inhibit TNF-α, LPS induced monocyte IL-1β and IL-12, and partially inhibit I-L6 production. Specific immunomodulatory compounds are discussed below.

Specific examples of such immunomodulatory compounds, include, but are not limited to, cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), including, but not limited to, those disclosed in U.S. Pat. Nos. 5,635,517, 6,476,052, 6,555,554, and 6,403,613; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; and isoindole-imide compounds such as those described in U.S. patent application Ser. No. 09/972,487 filed on Oct. 5, 2001, U.S. patent application Ser. No. 10/032,286 filed on Dec. 21, 2001, and International Application No. PCT/US01/50401 (International Publication No. WO 02/059106). The entireties of each of the U.S. patents and U.S. patent application publications identified herein are incorporated herein by reference. Immunomodulatory compounds do not include thalidomide.

The amount of the bioactive compound coating or impregnating the collagen may vary, and will preferably depend upon the particular bioactive compound to be delivered, and the effect desired.

In various embodiments, the collagen biofabric may be coated with, or impregnated with, at least 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 100, 1250, 1500, 2000, 2500, 300, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 200000, 300000, 400000, 500000, 600000, 700000, 800000, 900000 or at least 1000000 nanograms of a bioactive compound. In another embodiment, the ocular plug of the invention may be coated with, or impregnated with, no more than 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 100, 1250, 1500, 2000, 2500, 300, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 200000, 300000, 400000, 500000, 600000, 700000, 800000, 900000 or at least 1000000 nanograms of a bioactive compound.

5.6.3. Conformation of Collagen Biofabric

The collagen biofabric may be formed into any shape or conformation that will facilitate its use in the methods of the invention. For example, the collagen biofabric can be formed in any shape or conformation that will facilitate culturing stem cells. In some embodiments, the collagen biofabric is in a culture plate and is shaped according to the culture plate. In other embodiments, the collagen biofabric is in a well of a microwell plate and is shaped according to the well of the microwell plate.

The collagen biofabric useful in the treatment methods of the invention may be provided to the end user either dry, or pre-wetted in a suitable physiologically-compatible, medically-useful liquid, such as a saline solution. In one embodiment, the solution comprises one or more bioactive compounds, as described in Section 5.6.2 above, without limitation.

5.6.4. Methods of Making Collagen Biofabric

Collagen biofabric, made from amniotic membrane, chorionic membrane, or both, may be produced by any means that preserves the biochemical and structural characteristics of the membrane's components—chiefly collagen, elastin, laminin, and fibronectin. A preferred material is the collagen biofabric described in, and produced according to the methods disclosed in, United States Application Publication No. U.S. 2004/0048796 A1, “Collagen Biofabric and Methods of Preparation and Use Therefor” by Hariri, which is hereby incorporated herein in its entirety.

Preferably, the collagen biofabric used in stem cell culture is from a human placenta for use in human subjects, though the collagen biofabric may be made from amniotic membrane from a non-human mammal. Where the collagen biofabric is to be used in a non-human animal, it is preferred that the collagen biofabric be derived from a placenta from that species of animal.

In a preferred embodiment, the placenta for use in the methods of the invention is taken as soon as possible after delivery of the newborn. The placenta may be used immediately, or may be stored for 2-5 days from the time of delivery prior to any further treatment. The placenta is typically exsanguinated, that is, drained of the cord blood remaining after birth. Preferably, the expectant mother is screened prior to the time of birth, using standard techniques known to one skilled in the art, for communicable diseases including but not limited to, HIV, HBV, HCV, HTLV, syphilis, CMV, and other viral pathogens known to contaminate placental tissue.

One exemplary method for preparing a collagen biofabric of the invention comprises the following steps:

Step I.

The umbilical cord is separated from the placental disc; optionally, the amniotic membrane is separated from the chorionic membrane. In a preferred embodiment, the amniotic membrane is separated from the chorionic membrane prior to cutting the placental membrane. Following separation of the amniotic membrane from the chorionic membrane and placental disc, the umbilical cord stump is cut, e.g., with scissors, and detached from the placental disc. The amniotic membrane may then be stored in a sterile, preferably buffered, saline solution, such as 0.9% sterile NaCl solution. Preferably, the amniotic membrane is stored by refrigeration, at a temperature of at least 2° C.

Step II.

The amniotic membrane is substantially decellularized; that is, substantially all cellular material and cellular debris (e.g., all visible cellular material and cellular debris) is removed. Any decellularizing process known to one skilled in the art may be used, however, generally the process used for decellularizing the amniotic membrane of the invention does not disrupt the native conformation of the proteins making up the biofabric. “Substantial decellularization” of the amniotic membrane preferably removes at least 90% of the cells, more preferably removes at least 95% of the cells, and most preferably removes at least 99% of the cells (e.g., fibroblasts, amniocytes and chorionocytes). The amniotic membranes decellularized in accordance with the methods of the invention are uniformly thin, with inherent thickness variations of between about 2 and about 150 microns in the dry state, smooth (as determined by touch) and clear in appearance. Decellularization may comprise physical scraping, for example, with a sterile cell scraper, in combination with rinsing with a sterile solution. The decellularization technique employed should not result in gross disruption of the anatomy of the amniotic membrane or alter the biomechanical properties of the amniotic membrane. Preferably, the decellularization of the amniotic membrane comprises use of a detergent-containing solution, such as nonionic detergents, Triton X-100, anionic detergents, sodium dodecyl sulfate, Any mild anionic detergent, i.e., a non-caustic detergent, with a pH of 6 to 8, and low foaming, can be used to decellularize the amniotic membrane. In a specific embodiment, 0.01-1% deoxycholic acid sodium salt monohydrate is used in the decellularization of the amniotic membrane.

It is highly preferable to limit the protease activity in preparation of the biofabric. Additives to the lysis, rinse and storage solutions such as metal ion chelators, for example 1,10-phenanthroline and ethylenediaminetetraacetic acid (EDTA), create an environment unfavorable to many proteolytic enzymes. Providing sub-optimal conditions for proteases such as collagenase, assists in protecting amniotic membrane components such as collagen from degradation during the cell lysis step. Suboptimal conditions for proteases may be achieved by formulating the hypotonic lysis solution to eliminate or limit the amount of calcium and zinc ions available in solution. Many proteases are active in the presence of calcium and zinc ions and lose much of their activity in calcium and zinc ion free environments. Preferably, the hypotonic lysis solution will be prepared selecting conditions of pH, reduced availability of calcium and zinc ions, presence of metal ion chelators and the use of proteolytic inhibitors specific for collagenase such that the solution will optimally lyse the native cells while protecting the underlying amniotic membrane from adverse proteolytic degradation. For example a hypotonic lysis solution may include a buffered solution of water, pH 5.5 to 8, preferably pH 7 to 8, free from calcium and zinc ions and including a metal ion chelator such as EDTA. Additionally, control of the temperature and time parameters during the treatment of the amniotic membrane with the hypotonic lysis solution may also be employed to limit the activity of proteases.

It is preferred that the decellularization treatment of the amniotic membrane also limits the generation of new immunological sites. Since enzymatic degradation of collagen is believed to lead to heightened immunogenicity, the invention encompasses treatment of the amniotic membrane with enzymes, e.g., nucleases, that are effective in inhibiting cellular metabolism, protein production and cell division, that minimize proteolysis of the compositions of the amniotic membrane thus preserving the underlying architecture of the amniotic membrane. Examples of nucleases that can be used in accordance with the methods of the invention are those effective in digestion of native cell DNA and RNA including both exonucleases and endonucleases. A non-limiting example of nucleases that can be used in accordance with the methods of the invention include exonucleases that inhibit cellular activity, e.g., DNase I (SIGMA Chemical Company, St. Louis, Mo.) and RNase A (SIGMA Chemical Company, St. Louis, Mo.) and endonucleases that inhibit cellular activity, e.g., EcoRI (SIGMA Chemical Company, St. Louis, Mo.) and HindIII (SIGMA Chemical Company, St. Louis, Mo.). It is preferable that the selected nucleases are applied in a physiological buffer solution which contains ions, e.g., magnesium, calcium, which are optimal for the activity of the nuclease. Preferably, the ionic concentration of the buffered solution, the treatment temperature and the length of treatment are selected by one skilled in the art by routine experimentation to assure the desired level of nuclease activity. The buffer is preferably hypotonic to promote access of the nucleases to cell interiors.

In another embodiment of Steps I and II, above, the placenta, after initial processing, is briefly rinsed in saline to remove blood from the placental surface. The placental disk is then immersed in a cold deoxycholic acid solution at a concentration of about 0.1% to about 10%, and, in a specific embodiment, about 0.1% to about 2.0%. The placenta is then incubated in this solution at between about 1° C. to about 8° C. for about 5 days to about 6 months. In specific embodiments, the placental disk is immersed, for example, for about 5 to about 15 days; about 5 to about 30 days, about 5 to about 60 days, or for up to about one year. Typically, the deoxycholic acid solution is replaced during incubation every 2-5 days. In another specific embodiment, the placental disk is immersed in a deoxycholic acid solution at a concentration of about 1% at a temperature of 0° C. to about 8° C. for about 5 days to about 15 days. This incubation serves two purposes. First, it allows time for serological tests to be performed on the placental material and blood, so that placentas failing to meet serological criteria are not processed further. Second, the longer incubation improves the removal of epithelial cells and fibroblasts, which allows for a significant reduction in the amount of time spent decellularizing the amnion by physically scraping. Typically, the scraping time is reduced from, e.g., about 40 minutes to about 20 minutes. The amniotic membrane is then dried as described below.

Step III. Following decellularization, the amniotic membrane is washed to assure removal of cellular debris which may include cellular proteins, cellular lipids, and cellular nucleic acids, as well as any extracellular debris such as extracellular soluble proteins, lipids and proteoglycans. The wash solution may be de-ionized water or an aqueous hypotonic buffer. Preferably, the amniotic membrane is gently agitated for 15-120 minutes in the detergent, e.g., on a rocking platform, to assist in the decellularization. The amniotic membrane may, after detergent decellularization, again be physically decellularized as described above; the physical and detergent decellularization steps may be repeated as necessary, as long as the integrity of the amniotic membrane is maintained, until no visible cellular material and cellular debris remain.

In certain embodiments, the amniotic membrane is dried immediately (i.e., within 30 minutes) after the decellularization and washing steps. Alternatively, when further processing is not done immediately, the amniotic membrane may be refrigerated, e.g., stored at a temperature of about 1° C. to about 20° C., preferably from about 2° C. to about 8° C., for up to 28 days prior to drying. When the decellularized amniotic membrane is stored for more than three days but less than 28 days, the sterile solution covering the amniotic membrane is preferably changed periodically, e.g., every 1-3 days.

In certain embodiments, when the amniotic membrane is not refrigerated after washing, the amniotic membrane is washed at least 3 times prior to proceeding to Step IV of the preparation. In other embodiments, when the amniotic membrane has been refrigerated and the sterile solution has been changed once, the amniotic membrane is washed at least twice prior to proceeding to Step IV of the preparation. In yet other embodiments, when the amniotic membrane has been refrigerated and the sterile solution has been changed twice or more, the amniotic membrane is washed at least once prior to proceeding to Step IV of the preparation.

Prior to proceeding to Step IV, it is preferred that all bacteriological and serological testing be assessed to ensure that all tests are negative.

Step IV. The final step in this embodiment of the method of collagen biofabric production comprises drying the decellularized amniotic membrane of the invention to produce the collagen biofabric. Any method of drying the amniotic membrane so as to produce a flat, dry sheet of collagen may be used. Preferably, however, the amniotic membrane is dried under vacuum.

In a specific embodiment, an exemplary method for drying the decellularized amniotic membrane of the invention comprises the following steps:

Assembly of the Decellularized Amniotic Membrane for Drying.

The decellularized amniotic membrane is removed from the sterile solution, and the excess fluid is gently squeezed out. The decellularized amniotic membrane is then gently stretched until it is flat with the fetal side faced in a downward position, e.g., on a tray. The decellularized amniotic membrane is then flipped over so that fetal side is facing upwards, and placed on a drying frame, preferably a plastic mesh drying frame (e.g., QUICK COUNT® Plastic Canvas, Uniek, Inc., Waunakee, Wis.). In other embodiments, the drying frame may be any autoclavable material, including but not limited to a stainless steel mesh. In a most preferred embodiment, about 0.5 centimeter of the amniotic membrane overlaps the edges of the drying frame. In certain embodiments, the overlapping amniotic membrane extending beyond the drying frame is wrapped over the top of the frame, e.g., using a clamp or a hemostat. Once the amniotic membrane is positioned on the drying frame, a sterile gauze is placed on the drying platform of a heat dryer (or gel-dryer) (e.g., Model 583, Bio-Rad Laboratories, Hercules, Calif.), so that an area slightly larger than the amniotic membrane resting on the plastic mesh drying frame is covered. Preferably, the total thickness of the gauze layer does not exceed the thickness of one folded 4×4 gauze. Any heat drying apparatus may be used that is suitable for drying sheet like material. The drying frame is placed on top of the gauze on the drying platform so that the edges of the plastic frame extend above beyond the gauze edges, preferably between 0.1-1.0 cm, more preferably 0.5-1.0 cm. In a most preferred embodiment, the drying frame having the amniotic membrane is placed on top of the sterile gauze with the fetal side of the amniotic membrane facing upward. In some embodiments, another plastic framing mesh is placed on top of the amniotic membrane. In another embodiments, a sheet of thin plastic (e.g., SW 182, clear PVC, AEP Industries Inc., South Hackensack, N.J.) or a biocompatible silicone is placed on top of the membrane covered mesh so that the sheet extends well beyond all of the edges. In this embodiment, the second mesh frame is not needed.

In an alternative embodiment, the amniotic membrane is placed one or more sterile sheets of TYVEK® material (e.g., a sheet of TYVEK® for medical packaging, DuPont TYVEK®, Wilmington, Del.), optionally, with one sheet of TYVEK® on top of the membrane (prior to placing the plastic film). This alternate process will produce a smoother version of the biofabric (i.e., without the pattern of differential fiber compression regions along and perpendicular to the axis of the material), which may be advantageous for certain applications, such as for example for use as a matrix for expansion of cells.

Drying the amniotic membrane. In a preferred embodiment, the invention encompasses heat drying the amniotic membrane of the invention under vacuum. While the drying under vacuum may be accomplished at any temperature from about 0° C. to about 60° C., the amniotic membrane is preferably dried at between about 35° C. and about 50° C., and most preferably at about 50° C. It should be noted that some degradation of the collagen is to be expected at temperatures above 50° C. The drying temperature is preferably set and verified using a calibrated digital thermometer using an extended probe. Preferably, the vacuum pressure is set to about −22 inches of Hg. The drying step is continued until the collagen matrix of the amniotic membrane is substantially dry, that is, contains less than 20% water by weight, and preferably, about 3-12% water by weight as determined for example by a moisture analyzer. To accomplish this, the amniotic membrane may be heat-vacuum dried, e.g., for approximately 60 minutes to achieve a dehydrated amniotic membrane. In some embodiments, the amniotic membrane is dried for about 30 minutes to 2 hours, preferably about 60 minutes. Although not intending to be bound by any mechanism of action, it is believed that the low heat setting coupled with vacuum pressure allows the amniotic membrane to achieve the dehydrated state without denaturing the collagen.

After completion of the drying process in accordance with the invention, the amniotic membrane is cooled down for approximately two minutes with the vacuum pump running.

Packaging and Storing of the Amniotic Membrane.

Once the amniotic membrane is dried, the membrane is gently lifted off the drying frame. “Lifting off” the membrane may comprise the following steps: while the pump is still running, the plastic film is gently removed from the amniotic membrane starting at the corner, while holding the amniotic membrane down; the frame with the amniotic membrane is lifted off the drying platform and placed on a cutting board with the amniotic membrane side facing upward; an incision is made, cutting along the edge 1-2 mm away from the edge of the frame; the amniotic membrane is then peeled off the frame. Preferably, handling of the amniotic membrane at this stage is done with sterile gloves.

The amniotic membrane can placed in a sterile container, e.g., peel pouch, and sealed. In other embodiments, at least a portion of the collagen biofabric is subdivided into pieces suitable for placing in a culture dish or multiwell plate. For example, one or more circular pieces of collagen biofabric can be placed into a culture dish or multiwell plate such that the collagen biofabric covers at least a portion of the bottom (e.g., culturing surface) of the culture dish or multiwell plate. In a preferred embodiment, the entire circular culturing surface of a Petri dish, or circular culturing surface of one or more wells of a multiwell plate are completely covered by collagen biofabric.

The biofabric produced in accordance with the methods of the invention, either alone or in conjunction with a type of tissue culture dish or multiwell plate, may be stored at room temperature for an extended period of time as described supra.

In alternative embodiments, the collagen biofabric can comprise a chorionic membrane, or both a chorionic membrane and an amniotic membrane. It is expected that the methods described above would be applicable to the method of preparing a biofabric comprising a chorionic membrane, or both a chorionic membrane and an amniotic membrane. In one embodiment, the invention encompasses the use of a collagen biofabric prepared by providing a placenta comprising an amniotic membrane and a chorionic membrane; separating the amniotic membrane from the chorionic membrane; and decellularizing the chorionic membrane. In a specific embodiment, the preparation of the biofabric further entails washing and drying the decellularized chorionic membrane. In another embodiment, the invention encompasses the use of a collagen biofabric prepared by providing a placenta comprising an amniotic membrane and a chorionic membrane, and decellularizing the amniotic and chorionic membranes. In a specific embodiment, the method further entails washing and drying the decellularized amniotic and chorionic membranes.

5.6.5. Storage and Handling of Collagen Biofabric

Dehydrated collagen biofabric may be stored, e.g., as dehydrated sheets, at room temperature (e.g., 25° C.) prior to use. In certain embodiments, the collagen biofabric can be stored at a temperature of at least 10° C., at least 15° C., at least 20° C., at least 25° C., or at least 29° C. Preferably, collagen biofabric, in dehydrated form, is not refrigerated. In some embodiments, the collagen biofabric may be refrigerated at a temperature of about 2° C. to about 8° C. The biofabric produced according to the methods of the invention can be stored at any of the specified temperatures for 12 months or more with no alteration in biochemical or structural integrity (e.g., no degradation), without any alteration of the biochemical or biophysical properties of the collagen biofabric. The biofabric can be stored for several years with no alteration in biochemical or structural integrity (e.g., no degradation), without any alteration of the biochemical or biophysical properties of the collagen biofabric. The biofabric may be stored in any container suitable for long-term storage. Preferably, the collagen biofabric of the invention is stored in a sterile double peel-pouch package.

The collagen biofabric is typically hydrated prior to culturing, expanding, or differentiating a stem cell, e.g., an embryonic stem cell. The collagen biofabric can be rehydrated using, e.g., a sterile physiological buffer. In a specific embodiment, the sterile saline solution is a 0.9% NaCl solution. In some embodiments the sterile saline solution is buffered. Preferably, prior to culturing, expanding or differentiating a stem cell, the collagen biofabric is rehydrated in culture medium, e.g., DMEM, a stem cell culture medium, or the culture medium described in Section 4.2.1. In certain embodiments, the hydration of the collagen biofabric of the invention requires at least 2 minutes, at least 5 minutes, at least 10 minutes, at least 15 minutes, or at least 20 minutes. In a preferred embodiment, the hydration of the collagen biofabric of the invention is complete within 5 minutes. In yet another preferred embodiment, the hydration of the collagen biofabric of the invention is complete within 10 minutes. In yet another embodiment, the hydration of the collagen biofabric of the invention takes no more than 10 minutes. Once hydrated, the collagen biofabric may be maintained in solution, e.g., sterile 0.9% NaCl solution, for up to six months, with a change of solution, e.g., every three days.

5.6.6. Sterilization

Sterilization of the biofabric may be accomplished by any medically-appropriate means, preferably means that do not significantly crosslink or denature membrane proteins. Sterilization may be accomplished, for example, using gas, e.g., ethylene dioxide. Sterilization may be accomplished using radiation, for example, gamma radiation, and is preferably done by electron beam irradiation using methods known to one skilled in the art, e.g., Gorham, D. Byrom (ed.), 1991, Biomaterials, Stockton Press, New York, 55-122. Any dose of radiation sufficient to kill at least 99.9% of bacteria or other potentially contaminating organisms is within the scope of the invention. In a preferred embodiment, a dose of at least 18-25 kGy is used to achieve the terminal sterilization of the biofabric.

5.6.7. Laminates

The invention further provides the culture, expansion or differentiation of a stem cell, comprising culturing the cell in a culture medium with a laminate of a collagen biofabric. Such a laminate can be substantially flat (e.g., suitable for cell culture) or three-dimensional.

Collagen biofabric is typically laminated by stacking 2 or more layers of collagen biofabric one atop the other and sealing or drying. The collagen biofabric may be laminated either dry or after rehydration. Alternatively, two or more layers of, e.g., amniotic membrane may be laminated prior to initial drying after cell removal, e.g., via a cell scraping step (see Examples, below). If laminated prior to the initial drying, 2 or more collagen biofabric layers may be stacked one atop the other and subsequently dried, using, for example, a freeze-drying process, or drying under moderate heat with or without vacuum. The heat applied preferably is not so intense as to cause breakdown or decomposition of the protein components, especially the collagen, of the collagen biofabric. Typically, the heat applied is no more than about 70° C., preferably no more than about 60° C., and, more preferably, is approximately 50° C. Lamination time varies with, e.g., the number of layers being laminated, but typically takes 1-2 hours at 50° C. for the size pieces of collagen biofabric used for tympanic membrane repair.

The collagen biofabric may also be laminated using an adhesive applied between 2 or more layers of collagen biofabric or amniotic membrane. Such an adhesive is preferably appropriate for medical applications, and can comprise a natural biological adhesive, for example fibrin glue, a synthetic adhesive, or combinations thereof. The adhesive may further be chemically converted from precursors during the lamination process.

### 5.7. Kits

Collagen biofabric, useful for the methods of the present invention, may be provided in a wrapping or container as part of a kit for the facilitation of culturing, expanding or differentiating stem cells.

In one embodiment, the kit comprises one or more culture dishes or microwell plates, wherein said dishes or plates comprise collagen biofabric. In some embodiments, each piece of the collagen biofabric is provided in a culture dish or in each well of a microwell plate. In another embodiment, the kit comprises two or more pieces of collagen biofabric, separately wrapped or contained.

In another embodiment, the kit comprises medium suitable for the culture of a stem cell. In another embodiment the kit comprises one or more compounds that cause a stem cell to differentiate into an adult cell.

### 5.8. Uses of Stem Cells Cultured on Collagen Biofabric

The stem cells cultured, expanded, differentiated in accordance with the present invention have a variety of applications. The stem cells can be used for any purpose known by those of skill in the art, e.g., as described in U.S. Application Publication No. 2004/0048796, the contents of which is incorporated by reference in its entirety. For example, stem cells can be used in transplantation and ex vivo treatment protocols in which a tissue or organ of the body is augmented, repaired or replaced by the engraftment, transplantation or infusion of a desired cell population, such as a stem cell or progenitor cell population. They can also be used to replace or augment existing tissues, to introduce new or altered tissues, or to join together biological tissues or structures. The stem cell culture with the collagen biofabric can also be used in surgical procedures, for instance, as a surgical graft.

Stem cells that have been cultured on collage biofabric can be used without the collagen biofabric. That is, the stem cells can be separated from the collagen biofabric by methods know to those of skill in the art, e.g., removal by trypsinization and washing. These stem cells can then be used for further stem cell culture, or to treat a disease, disorder or condition that is treatable using stem cells. In another embodiment, the stem cells can be used with the collagen biofabric in any application in which collagen biofabric can be used to treat a disease, disorder or condition. See., e.g., Hariri et al., U.S. Application Publication No. 20040048796, Hariri & Smiell, U.S. Provisional Application No. 60/699,441, filed Jul. 13, 2005; Lin & Ray, U.S. Provisional Application No. 60/699,440, filed Jul. 13, 2005; and Sulner et al., U.S. Provisional Application No. 60/696,197, filed Jun. 30, 2005. In another embodiment, stem cells differentiated on collagen biofabric (e.g., adult cells) can be used without the collagen biofabric in tissue-appropriate applications (e.g., stem cells differentiated into cardiac cells can be used to repair tissue damaged in a cardiac infarct). In another embodiment, differentiated stem cells can be used with the collagen biofabric on which they were differentiated in tissue-appropriate applications (e.g., stem cells differentiated to chondrocytes can be used with the collagen biofabric, e.g., to repair a damaged joint).

5.9. Methods of Screening Compounds

The present invention provides methods of screening for compounds that modulate the expansion or differentiation of stem cells, or modulate the activity of cells. The compounds to be screened can be small molecules, drugs, peptides, polynucleotides, etc., or libraries of such candidate compounds. The cell can be a somatic cell or stem cell. The cell can be a naturally occurring cell or a cell engineered to express a recombinant gene product. In the context of stem cells, since the collage biofabric can replace the feeder cells in culture, the methods have the advantage of not being complicated by a secondary effect caused by perturbation of the feeder cells of the test compound.

In one aspect, the present invention provides a method for determining the toxicity of a compound to a cell, using the collagen biofabric cell culture system of the invention. In some embodiments, the method comprises culturing said cell with a collagen biofabric under conditions suitable for the survival of the cell, contacting the cell with a compound, and detecting apoptosis, necrosis, or cell death, or a tendency towards apoptosis, necrosis or cell death. If apoptosis, necrosis, cell death, or a tendency towards the same is detected, compared to a cell not contacted with the compound, said compound is toxic to said cell. In a specific embodiment, said cell is a part of a plurality of said stem cells, wherein each of said stem cells is contacted with one of a plurality of compounds to identify a subset of said plurality of compounds that have an effect on apoptosis or cell death.

In another aspect, the present invention provides methods for determining the effect of a compound on the differentiation of a stem cell, e.g., using the collagen biofabric cell culture system of the invention. In some embodiments, the methods comprise culturing said cell with a collagen biofabric under conditions suitable for the differentiation of the cell. The cell is contacted with a compound. The cells are then analyzed for a marker of the differentiation in the presence of absence of the candidate compound. The marker of the differentiation can be a cell surface marker, cell morphology or one or more differentially expressed genes. If a change is identified, said compound has an effect on the differentiation of said cell. In a specific embodiment, said cell is a part of a plurality of said stem cells, wherein each of said stem cells is contacted with one of a plurality of compounds to identify a subset of said plurality of compounds that have an effect on differentiation of said stem cell.

### 6. EXAMPLES

### 6.1. Example 1: Method of Making Collagen Biofabric Materials

The following materials were used in preparation of the collagen biofabric.

### Materials/Equipment

Copy of Delivery Record
Copy of Material/Family Health History/Informed Consent
Source Bar Code Label (Donor ID number)
Collection # (A sequential number is assigned to incoming material)
Tissue Processing Record (Document ID #ANT-19F); a detailed record of processing of each lot number is maintained
Human Placenta (less than 48 hours old at the start of processing)
Sterile Surgical Clamps/Hemostats
Sterile Scissors
Sterile Scalpels
Sterile Steri-Wrap sheets
Sterile Cell Scraper (Nalgene NUNC Int. R0896)
Sterile Gauze (non-sterile PSS 4416, sterilized)
Sterile Rinsing Stainless Steel Trays
Disinfected Processing Stainless Steel Trays
Disinfected Plastic Bin
Sterile 0.9% NaCl Solution (Baxter 2F7124)
Sterile Water (Milli Q plus 09195 or Baxter 2F7113)
Sterile Specimen Containers (VWR 15704-014)
Personal Protective Equipment (including sterile and non-sterile gloves)
Certified Clean Room
Previously Prepared Decellularizing Solution (D-cell); 0.01-1% deoxycholic acid sodium monohydrate
Disinfected Bin
Rocking Platform (VWR Model 100)
Timer (VWR 21376890)
Disinfected Plastic Frame Mesh
PVC Wrap Film
Vacuum Pump (Schuco-Vac 5711-130)
Gel Dryer (i.e., heat dryer; BioRad Model 583)
Disinfected Stainless Steel Cutting Board
Pouches for Packaging
Sterile Stainless Steel Ruler (General Tools MFG. Co 1201)
Traceable Digital Thermometer (Model 61161-364, Control Company)
Accu-Seal Automatic Sealer (Accu-Seal, Model 630-1B6)

The expectant mother was screened at the time of birth for communicable diseases such as HIV, HBV, HCV, HTLV, syphilis, CMV and other viral and bacterial pathogens that could contaminate the placental tissues being collected. Only tissues collected from donors whose mothers tested negative or non-reactive to the above-mentioned pathogens were used to produce the collagen biofabric.

Following normal birth, the placenta, umbilical cord and umbilical cord blood were spontaneously expelled from the contracting uterus. The placenta, umbilical cord, and umbilical cord blood were collected following birth. The materials were transported to the laboratory where they were processed under aseptic conditions in a Clean room having a HEPA filtration system, which was turned on at least one hour prior to processing. Gloves (sterile or non-sterile, as appropriate) were worn at all times while handling the product. All unused (waste) segments of the amnion/chorion and contaminated liquids generated during tissue processing were disposed of as soon as feasible.

Step I.

A sterile field was set up with sterile Steri-Wrap sheets and the following instruments and accessories for processing were placed on it.
sterile tray pack
sterile Cell Scraper
sterile scalpel
disinfected processing tray

Sterile pack ID # was recorded in the Processing Record.

The placenta was removed from the transport container and placed onto the disinfected stainless steel tray. Using surgical clamps and scissors, the umbilical cord was cut off approximately 2 inches from the placental disc. The umbilical cord was placed into a separate sterile container for further processing. The container was labeled with Tissue ID Bar Code; and the material and storage solution(s) present (e.g., type of media) were identified. In some cases, the umbilical cord was discarded if not requested for other projects.

Starting from the edge of the placental membrane, the amnion was separated from the chorion using blunt dissection with fingers. This was done prior to cutting the membrane.

After the amnion was separated from the entire surface of the chorion and placental disc, the amniotic membrane was cut around the umbilical cord stump with scissors and detached from the placental disc. In some instances, if the separation of the amnion and chorion was not possible without tearing the tissue, the amnion and chorion were cut from the placental disc as one piece and then peeled apart.

The chorion was placed into a separate specimen container to be utilized for other projects. The container was labeled with the Tissue ID Bar Code, the material and storage solution(s) present (e.g., type of media) were identified, initialed and dated.

If any piece of amnion was still attached to the placental disc it was peeled from the disc and cutting off around the umbilical cord with scissors. The placenta was placed back into the transport container to be utilized for other projects.

The appropriate data was recorded in the Tissue Processing Record.

The amniotic membrane was kept in the tray with sterile 0.9% NaCl solution. Preferably, the amniotic membrane is stored by refrigeration for a maximum of 72 hours from the time of delivery prior to the next step in the process.

Step II.

The amniotic membrane was removed from the specimen container one piece at a time and placed onto the disinfected stainless steel tray. Other pieces were placed into a separate sterile stainless steel tray filled with sterile water until they were ready to be cleaned. Extra pieces of amnion from the processing tray were removed and placed in a separate rinsing stainless steel tray filled with sterile water.

The amniotic membrane was rinsed with sterile water if grossly contaminated with blood maternal or fetal fluids/materials changing sterile water as needed.

The amniotic membrane was placed on the processing tray with the maternal side facing upward. Using a sterile Cell Scraper, as much as possible of visible contamination and cellular material from the maternal side of the amnion was carefully removed. (Note: minimal pressure should be applied for this step to prevent tearing the membrane). Sterile water was used to aid in the removal of cells and cellular debris. The amniotic membrane was further rinsed with sterile water in the separate sterile stainless steel rinsing tray.

The amniotic membrane was turned over so that the fetal side was facing upward and placed back on the processing tray and rinsed with sterile water. Visible cellular material and debris using the Cell Scraper was gently removed (Note: minimal pressure should be applied for this step to prevent tearing the membrane). Sterile water was used to aid in the removal of cells and cellular debris.

The amniotic membrane was rinsed with sterile water in between cleaning rounds in separate sterile rinsing trays. The tissue was cleaned as many times (cleaning rounds) as necessary to remove most if not all of visible cellular material and debris from both sides of the membrane. The sterile water was changed in the rinsing trays in between rinses.

The processing tray was rinsed with sterile water after each cleaning round.

All other pieces of amnion were processed in the same manner and placed into the same container. Tissue Id Bar Code was affixed, the material and storage solution(s) present (e.g., type of media) were identified, initials date were added.

The appropriate information and the date were recorded in the Tissue Processing Record.

Step III.

The amniotic membrane was removed from the rinsing tray, (or from storage container) excess fluid was gently squeezed out with fingers and the membrane was placed into the sterile specimen container. The container was filled up to the 150 ml mark with D-cell solution ensuring that all of the amniotic membrane was covered and the container was closed.

The container was placed in the bin on the rocking platform. The rocking platform was turned on and the membrane was agitated in D-cell solution for a minimum of 15 minutes and a maximum of 120 minutes at Setting #6.

A new sterile field was set up with new sterile instruments and disinfected tray in a same manner as in the Step I. Sterile pack ID # was recorded in the Processing Record.

After agitation was completed, the rocking platform was turned off and the membrane was removed from the container. The membrane was placed into a new sterile stainless steel processing tray. Sterile 0.9% NaCl solution was added to cover the bottom of the tray.

Using a new sterile Cell Scraper, residual D-cell and cellular material (if any) was removed from both sides of the tissue. This step was repeated as many times as needed to remove as much as possible of visible residual cellular material from the entire surface on both sides. The membrane was rinsed with sterile 0.9% NaCl solution in a separate rinsing tray in between cleaning rounds. The sterile 0.9% NaCl solution was changed in the rinsing trays in between rinses.

After the last cleaning round was completed, the membrane was rinsed with sterile 0.9% NaCl solution and placed into the new sterile specimen container filled with sterile 0.9% NaCl solution.

All remaining pieces of amniotic membrane were processed in exactly the same manner.

When all amniotic membrane pieces were processed and in the container with the sterile 0.9% NaCl solution, the container was placed in the bin on the rocking platform to agitate for a minimum of 5 minutes at setting #6. After agitation was completed, the membrane was removed from the specimen container, the sterile 0.9% NaCl solution was changed in the container and the membrane was placed back into the specimen container.

The specimen container was labeled with Tissue ID Bar Code and Quarantine label. The material and storage solution(s) present (e.g., type of media) were identified, initialed and dated. The specimen container was placed into a clean zip-lock bag and placed in the refrigerator (2-8° C.).

All appropriate data was recorded in the Tissue Processing Record.

When serology results became available, the appropriate label (Serology Negative or For Research Use Only) was placed on the top of the Quarantine label and those containers were segregated from Quarantined ones.

Step IV.

Before proceeding with Step IV, the Tissue Status Review was checked to make sure all applicable test results were negative.

A sterile field was set up with sterile Steri-Wrap sheet and all sterile and disinfected instruments and accessories were set up in the same manner as in Steps II and III.

The membrane was removed from the refrigerator and placed into a new sterile stainless steel processing tray. Sterile 0.9% NaCl solution was added to cover the bottom of the tray.

All visible cellular material and debris (if any) was gently removed using a new sterile Cell Scraper (Note: minimal pressure should be applied for this step to prevent tearing the membrane). Sterile 0.9% NaCl solution was used to aid in removal of the cells and debris.

The membrane was rinsed in the separate sterile stainless steel rinsing tray filled with the sterile 0.9% NaCl Solution. 0.9% NaCl Solution was changed in between cleaning rounds. The membrane was placed into a new sterile specimen container, the container was filled with fresh sterile 0.9% NaCl solution and placed on the rocking platform for agitation for a minimum of 5 minutes at Setting #6.

The previous step was repeated 3 times and the sterile 0.9% NaCl solution was changed in between each agitation. Appropriate data was recorded in the Tissue Processing Record.

The membrane was removed from the specimen container one piece at a time, excess fluid was gently squeezed out with fingers and the membrane was placed onto a sterile processing tray. The membrane was gently stretched until flat; ensuring it was fetal side down.

The frame was prepared by cutting the disinfected plastic sheet with sterile scissors. The size of the frame should be approximately 0.5 cm smaller in each direction than the membrane segment. The frame was rinsed in the rinsing tray filled with sterile 0.9% NaCl solution.

The frame was placed on the slightly stretched membrane surface and pressed on it gently. It is imperative that the smooth side of the plastic frame faces the tissue.

Using a scalpel, the membrane was cut around the frame leaving approximately 0.5 cm extending beyond frame edges. The excess membrane was placed back into the specimen container

The membrane edges that are extended beyond the frame were wrapped over the edges of the frame using clamps or tweezers and put aside on the same tray.

The next piece of membrane was processed in the same manner. It is preferred that the total area to be dried does not exceed 300 cm² per heat dryer. While ‘framing out’ the piece of membrane, it is preferred that the non-framed pieces remain in the container in sterile 0.9% NaCl solution.

The drying temperatures of dryers were set and verified using a calibrated digital thermometer with extended probe. The drying temperature was set at 50° C. The data was recorded in the Tissue Processing Record.

The vacuum pump was turned on.

A sterile gauze was placed on the drying platform of the heat dryer, covering an area slightly larger than the area of the framed membrane. It is important to make sure that the total thickness of the gauze layer does not exceed thickness of one folded 4×4 gauze.

One sheet of plastic framing mesh was placed on top of the gauze. The plastic mesh edges should extend approximately 0.5-1.0 cm beyond gauze edges.

The framed membrane was gently lifted and placed on the heat dryer platform on top of the plastic mesh with the membrane side facing upward. This was repeated until the maximum amount of membrane (without exceeding 300 cm²) was on the heat dryer platform. (NOTE: fetal side of the amnion is facing up).

A piece of PVC wrap film was cut large enough to cover the entire drying platform of the heat dryer plus an extra foot.

With the vacuum pump running, the entire drying platform of the heat dryer was gently covered with the plastic film leaving ½ foot extending beyond drying platform edges on both sides. Care was taken that the film pull tightly against the membrane and frame sheet (i.e., it is “sucked in” by the vacuum) and that there were no air leaks and no wrinkles over the tissue area). The lid was subsequently closed.

The vacuum pump was set to approximately −22 inches Hg of vacuum. The pump gage was recorded after 2-3 min of drying cycle. The membrane was heat vacuum dried for approximately 60 minutes. Approximately 15-30 minutes into the drying process, the sterile gauze layer was replaced in the heat dryer with a new one. The total thickness of the gauze layer must not exceed thickness of one folded 4×4 gauze.

After the change, care was taken so that the plastic film pulled tightly against the membrane and the frame sheet and there were no air leaks and no wrinkles over the membrane area.

The integrity of the vacuum seal was periodically checked by checking the pump pressure monometer. After completion of the drying process, the heat dryer was opened and the membrane was cooled down for approximately two minutes with the pump running.

A new sterile field was set up with sterile Steri-wrap and disinfected stainless steel cutting board underneath it. As this point sterile gloves were used. With the pump still running, the plastic film was gently removed from the membrane sheet starting at the corner and holding the membrane sheet down with a gloved hand. The frame was gently lifted with the membrane off the drying platform and placed on the sterile field on the top of the disinfected stainless steel cutting board with the membrane side facing upward. Using a scalpel, the membrane sheet was cut through making an incision along the edge 1-2 mm away from the edge of the frame. The membrane was held in place with a gloved (sterile glove) hand. Gently the membrane sheet was lifted off of the frame by peeling it off slowly and then placed on the sterile field on the cutting board.

Using scalpel or sharp scissors, the membrane sheet was cut into segments of specified size. All pieces were cut and secured on the sterile field before packaging. A single piece of membrane was placed inside the inner peel-pouch package with one hand (sterile) while holding the pouch with another hand (non-sterile). Care was taken not to touch pouches with ‘sterile’ hand. After all pieces were inside the inner pouches they were sealed. A label was affixed with the appropriate information (e.g., Part #, Lot #, etc.) in the designated area on the outside of the pouch. All pieces of membrane were processed in the same manner. The labeled and sealed peel-pouch packages were placed in the waterproof zip-lock bag for storage until they were ready to be shipped to the sterilization facility or distributor. All appropriate data were recorded on the Tissue Processing Record.

### 6.2. Example 2: Alternative Method of Making Collagen Biofabric

A placenta is prepared substantially as described in Step I of Example 1 using the Materials in that Example. An expectant mother is screened at the time of birth for communicable diseases such as HIV, HBV, HCV, HTLV, syphilis, CMV and other viral and bacterial pathogens that could contaminate the placental tissues being collected. Only tissues collected from donors whose mothers tested negative or non-reactive to the above-mentioned pathogens are used to produce the collagen biofabric.

A sterile field is set up with sterile Steri-Wrap sheets and the following instruments and accessories for processing were placed on it: sterile tray pack; rinsing tray, stainless steel cup, clamp/hemostats, tweezers, scissors, gauze.

The placenta is removed from the transport container and placed onto a disinfected stainless steel tray. Using surgical clamps and scissors, the umbilical cord is cut off approximately 2 inches from the placental disc.

Starting from the edge of the placental membrane, the amnion is separated from the chorion using blunt dissection with fingers. This is done prior to cutting the membrane. After the amnion is separated from the entire surface of the chorion and placental disc, the amniotic membrane is cut around the umbilical cord stump with scissors and detached from the placental disc. In some instances, if the separation of the amnion and chorion is not possible without tearing the tissue, the amnion and chorion is cut from the placental disc as one piece and then peeled apart.

The appropriate data is recorded in the Tissue Processing Record.

The amniotic membrane is rinsed with sterile 0.9% NaCl solution to remove blood and fetal fluid or materials. The saline solution is replaced as necessary during this rinse.

The amnion is then placed in a 0.9% saline, 1.0% deoxycholic acid solution in a specimen container and refrigerated at 2-8° C. for up to 15 days, with changes of the solution every 3-5 days. During or at the end of incubation, the serological tests noted above are evaluated. If the tests indicate contamination with one or more pathogens, the amnion is rejected and processed no further. Tissue indicated as derived from a CMV-positive donor, however, is still suitable for production of biofabric.

Once the incubation is complete, the amnion is removed from the specimen container, placed in a sterile tray and rinsed three times with 0.9% NaCl solution to reduce the deoxycholic acid from the tissue. With the amnion placed maternal side up, the amnion is gently scraped with a cell scraper to remove as much cellular material as possible. Additional saline is added as needed to aid in the removal of cells and cellular debris. This step is repeated for the fetal side of the amnion. Scraping is followed by rinsing, and is repeated, both sides, as many times as necessary to remove cells and cellular material. The scraped amnion is rinsed by placing the amnion in 0.9% saline solution a separate container on a rocking platform for 5-120 minutes at setting #6. The saline solution is replaced, and the rocking rinse is repeated.

After rinsing is complete, the amnion is optionally stored in a zip-lock bag in a refrigerator.

The scraped amnion is then placed fetal side down onto a sterile processing tray. The amnion is gently massaged by hand to remove excess liquid, and to flatten the membrane. A sterile plastic sheet is cut so that its dimensions are approximately 0.5 cm smaller in each direction than the flat amnion. This plastic sheet is briefly rinsed in 0.9% NaCl solution. The plastic sheet is placed, smooth side down, on the flattened amnion, leaving a margin of uncovered amnion. A scalpel is used to trim the amnion, leaving approximately 0.5 cm extending beyond the sheet edges. These extending amnion edges are wrapped back over the plastic sheet. The total tissue area to be dried does not exceed 300 cm² for a standard vacuum heat dryer.

A sheet of sterile gauze is placed in a vacuum heat dryer. A thin plastic mesh is placed on the gauze so that approximately 0.5-10.0 cm extends beyond the edges of the gauze. The amnion and plastic sheet are then placed into the vacuum heat dryer on top of the mesh, tissue side up, and the amnion is covered with a sheet of PVC wrap film. The dryer is set at 50° C., and the temperature is checked periodically to ensure maintenance of 50° C.±1° C. The vacuum pump is then turned on and set to approximately −22 inches Hg vacuum. Drying is allowed to proceed for 60 minutes.

The dried amnion is then stored in a sealed plastic container for further use.

### 6.3. Example 3: Collagen Biofabric Laminate

The collagen biofabric produced by the methods described above was laminated as follows. Dry collagen biofabric was, in some instances, rehydrated in sterile 0.9% NaCl solution for 1 hour, 10 minutes to 1 hour, 30 minutes. Dry collagen biofabric was produced by the entire procedure outlined above (Example 1), then laminated; wet collagen biofabric was prepared up to Step III, then laminated. After mounting frames were cut, the rehydrated tissue was mounted by placing the fetal side down, placing the mounting frame on top of the tissue, and cutting the tissue, leaving about 1 cm edge around the frame. The 1 cm edge was folded over the edge of the frame using a cell scraper. These steps were repeated for adding additional pieces of wet collagen biofabric. The laminated biofabric was then placed in a gel dryer and dried to substantial dryness (<20% water content by weight). Laminates were then cut to 2×6 cm samples.

Separate lots of the laminated collagen biofabric were evaluated as follows. Dimensions of dry (DT) and wet (WT) laminated collagen biofabric were determined for laminates containing 2, 3, 5 or 8 layers, as shown in Table 1:

TABLE I

| | Thickness | Length | Width | Weight |
|---|---|---|---|---|
| | (μm) | (mm) | (mm) | (mg) |
| DT2 | 29 ± 12 | 20.0 ± 0.3 | 5.2 ± 0.1 | 0.87 ± 0.02 |
| DT3 | 32 ± 2 | 20.5 ± 0.1 | 5.2 ± 0.2 | 1.26 ± 0.11 |
| WT2 | 20 ± 15 | 20.2 ± 0.2 | 5.0 ± 0.3 | 0.93 ± 0.17 |
| WT3 | 15 ± 5 | 19.6 ± 0.1 | 5.1 ± 0.3 | 0.9 ± 0.04 |
| WT5 | 31 ± 5 | 19.8 ± 0.4 | 5.3 ± 0.1 | 2.06 ± 0.2 |
| WT8 | 115 ± 26 | 20.3 ± 0.2 | 5.1 ± 0.4 | 4.92 ± 0.56 |

Specimens showed no signs of delamination over the first two days post-lamination, when kept under dry conditions at room temperature. The laminated collagen biofabric additionally showed no signs of delamination when kept in stirred 0.9% saline, room temperature, for ten days.

Larger laminated collagen biofabric specimens were tested for laminate durability and resistance to delamination. 1×2 cm specimens from the list listed above (i.e., DT2, DT3, WT2, WT3, WT5 and WT8) were placed in Petri dishes in 5 ml phosphate buffered saline. The specimens were left on an orbital shaker for approximately 24 hours at 95 RPM. No delamination of the specimens was observed, either during shaking or thereafter during simple handling.

### 6.4. Example 4: A Kit for Culturing Stem Cells Using Collagen Biofabric

This examples provides a kit for culturing, expanding or differentiating stem cells using collagen biofabric.

The kit comprises, in a sealed container, a plurality of microwell plates suitable for culturing, expanding or differentiating stem cells. The microwell plate may comprise six, twelve, twenty four, or ninety six wells for cell culture. In each well, a single sheet of collagen biofabric or a collagen biofabric laminate is provided. The collagen biofabric and a collagen biofabric laminate are produced and prepared as described in Examples 1-3 above.

The kit also comprises a set of instructions for culturing, expanding or differentiating stem cells. In addition, the kit comprises one or more containers of culture medium suitable for culturing, expanding or differentiating stem cells and one or more agents that facilitate the growth or differentiation of stem cells.

### 6.5. Example 5: Culturing, Expanding and Differentiating Human Placental Stem Cells Using Collagen Biofabric

This examples provides culturing, expanding or differentiating human placental stem cells using collagen biofabric.

Human placental stem cells as utilized herein are described in U.S. Application Publication No. 2003/032179. Such cells are OCT-4⁺ and ABC-p⁺. Human placental stem cells are obtained from a placenta following expulsion from the uterus. Briefly, a placenta is exsanguinated and perfused with a suitable aqueous perfusion fluid, such as an aqueous isotonic fluid in which an anticoagulant is dissolved. After exsanguination and a sufficient time of perfusion of the placenta, the placental stem cells are observed to migrate into the exsanguinated and perfused microcirculation of the placenta. After cultured in the placenta for sufficient time, the placental stem cells are collected by collecting the effluent perfusate in a collecting vessel. The placental cells collected from the placenta are recovered from the effluent perfusate using techniques known by those skilled in the art, such as, for example, density gradient centrifugation, flow cytometry, etc.

The placental stem cells are then cultured with collagen biofabric using the kit as provided in Example 4. About 1-5×10⁵ cells are plated on the collagen biofabric in each well of the microwell plate of the kit. 5 ml culture medium is added into each well. The culture medium comprises 60% DMEM-LG (Gibco), 40% MCDB-201(Sigma), 2% fetal calf serum (FCS) (Hyclone Laboratories), 1×insulin-transferrin-selenium (ITS), 1×lenolenic-acid-bovine-serum-albumin (LA-BSA), 10⁻⁹M dexamethasone (Sigma), 10⁻⁴M ascorbic acid 2-phosphate (Sigma), epidermal growth factor (EGF)10 ng/ml (R&D Systems), platelet derived-growth factor (PDGF-BB) 10 ng/ml (R&D Systems), and 100 U penicillin/1000 U streptomycin.

The microwell plate is cultured in an incubator at 37° C. in a humidified atmosphere with 5% CO₂ to allow the recovery and attachment of the cells. All culture medium is changed every two days.

Human placental stem cells are induced to differentiate into neurons as follows. Human placental stem cells are cultured with collagen biofabric using the kit in Example 4 for 24 hours in preinduction media consisting of DMEM/20% FBS and 1 mM beta-mercaptoethanol. Preinduction media is removed and cells are washed with PBS. Neuronal induction media consisting of DMEM and 1-10 mM betamercaptoethanol is added. Alternatively, induction media consisting of DMEM/2% DMSO/200 μM butylated hydroxyanisole may be used to enhance neuronal differentiation efficiency. In certain embodiments, morphologic and molecular changes may occur as early as 60 minutes after exposure to serum-free media and betamercaptoethanol (Woodbury et al., J. Neurosci. Res., 61:364-370). RT/PCR is used to detect the expression of nerve growth factor receptor and neurofilament heavy chain genes, which are indicative of neural differentiation. The cells are also examined for development of a neural phenotype, e.g., development of dendrites and/or an axon.

Human placental stem cells are induced to differentiate into adipocytes as follows. Human placental stem cells are cultured with collagen biofabric using the kit in Example 4 to 50-70% confluency are induced in medium comprising (1) DMEM/MCDB-201 with 2% FCS, 0.5% hydrocortisone, 0.5 mM isobutylmethylxanthine, 60 μM indomethacin; or (2) DMEM/MCDB-201 with 2% FCS and 0.5% linoleic acid. Cells are examined for morphological changes. Typically, oil droplets appear after 3-7 days. Differentiation is assessed by quantitative real-time PCR to examine the expression of specific genes associated with adipogenesis, i.e., PPAR-γ2, aP-2, lipoprotein lipase, and osteopontin.

Chondrogenic differentiation of placental stem cells is accomplished as follows. Placental stem cells are cultured with collagen biofabric using the kit in Example 4 in MSCGM (Cambrex) or DMEM supplemented with 15% cord blood serum. Placental stem cells are aliquoted into a sterile polypropylene tube. The cells are centrifuged (150×g for 5 minutes), and washed twice in Incomplete Chondrogenesis Medium (Cambrex). After the last wash, the cells are resuspended in Complete Chondrogenesis Medium (Cambrex) containing 0.01m/m1 TGF-beta-3 at a concentration of 5×10(5) cells/ml. 0.5 ml of cells is aliquoted into a 15 ml polypropylene culture tube. The cells are pelleted at 150×g for 5 minutes. The pellet is left intact in the medium. Loosely capped tubes are incubated at 37° C., 5% CO² for 24 hours. The cell pellets are fed every 2-3 days with freshly prepared complete chondrogenesis medium. Pellets are maintained suspended in medium by daily agitation using a low speed vortex. Chondrogenic cell pellets are harvested after 14-28 days in culture. Chondrogenesis is characterized by e.g., observation of production of esoinophilic ground substance, assessing cell morphology, an/or RT/PCR confirmation of collagen 2 and/or collagen 9 gene expression and/or the production of cartilage matrix acid mucopolysaccharides, as confirmed by Alcian blue cytochemical staining.

Osteogenic differentiation of placental stem cells is accomplished as follows. Placental stem cells are cultured with collagen biofabric using the kit in Example 4 in osteogenic medium. Osteogenic medium is prepared from 185 mL Cambrex Differentiation Basal Medium—Osteogenic and SingleQuots (one each of dexamethasone, 1-glutamine, ascorbate, pen/strep, MCGS, and β-glycerophosphate). Placental stem cells from perfusate are plated, at about 3×10³ cells per cm² of tissue culture surface area in 0.2-0.3 mL MSCGM per cm² tissue culture area. Typically, all cells adhere to the culture surface for 4-24 hours in MSCGM at 37° C. in 5% CO₂. Osteogenic differentiation is induced by replacing the medium with Osteogenic Differentiation medium. Cell morphology begins to change from the typical spindle-shaped appearance of the adherent placental stem cells, to a cuboidal appearance, accompanied by mineralization. Some cells delaminate from the tissue culture surface during differentiation.

Pancreatic differentiation of placental stem cells is accomplished as follows. Placental stem cells are cultured with collagen biofabric, using the kit in Example 4, in DMEM/20% CBS, supplemented with basic fibroblast growth factor, 10 ng/ml; and transforming growth factor beta-1, 2 ng/ml. KnockOut Serum Replacement may be used in lieu of CBS. Conditioned media from nestin-positive neuronal cell cultures is added to media at a 50/50 concentration. Cells are cultured for 14-28 days, refeeding every 3-4 days. Differentiation is characterized by assaying for insulin protein or insulin gene expression by RT/PCR.

Myogenic (cardiogenic) differentiation of placental stem cells is accomplished as follows. Placental stem cells are cultured with collagen biofabric, using the kit in Example 4, in DMEM/20% CBS, supplemented with retinoic acid, 1 μM; basic fibroblast growth factor, 10 ng/ml; and transforming growth factor beta-1, 2 ng/ml; and epidermal growth factor, 100 ng/ml. KnockOut Serum Replacement (Invitrogen, Carlsbad, Calif.) may be used in lieu of CBS. Alternatively, placental stem cells are cultured in DMEM/20% CBS supplemented with 50 ng/ml Cardiotropin-1 for 24 hours. Alternatively, placental stem cells are maintained in protein-free media for 5-7 days, then stimulated with human myocardium extract (escalating dose analysis). Myocardium extract is produced by homogenizing 1 gm human myocardium in 1% HEPES buffer supplemented with 1% cord blood serum. The suspension is incubated for 60 minutes, then centrifuged and the supernatant collected. Cells are cultured for 10-14 days, refeeding every 3-4 days. Differentiation is confirmed by demonstration of cardiac actin gene expression by RT/PCR.

### 6.6. Example 6: Culture of Placental Stem Cells and Fibroblasts on Amniotic Membrane

The main goal of tissue engineering is the regeneration of living tissues/organs for the replacement of diseased or lost tissue/organs. Amniotic membrane is an excellent scaffold for providing a natural microenvironment for cell attachment and differentiation.

Membrane Preparation.

Amniotic membrane, prepared as described herein, was derived from the placentas of normal, full-term pregnancies. The amniotic membrane product was decellularized using a combination of detergent soaking and mechanical scraping. The final product was dehydrated at mild temperature and terminally sterilized by radiation.

Cellular Assays.

Normal human dermal fibroblasts (Cambrex) or placental stem cells, obtained by enzymatic digestion as described elsewhere herein, were cultured on amniotic membranes, fibronectin-(Sigma) or VITROGEN™—(Bovine collagen from Cohesion) coated surfaces for 4 or 24 hours. Cultured cells on the substrates were fixed in formalin, and stained for F-actin.

Results.

At 4 hours after seeding, distinct morphologies were observed in response to the various surfaces. Fibroblasts on fibronectin were well spread, displaying actin stress fibers, characteristic of an adherent cellular phenotype, while fibroblasts on collagen were not as spread, but instead showed numerous filopodial projections. Fibroblasts on amniotic membrane appear morphologically very similar to cells cultured on fibronectin. By 24 hours, differences in cellular morphology between the various substrates are much less apparent. Placental stem cells attached to the experimental substrates and showed differential cellular morphologies similar to the fibroblasts′.

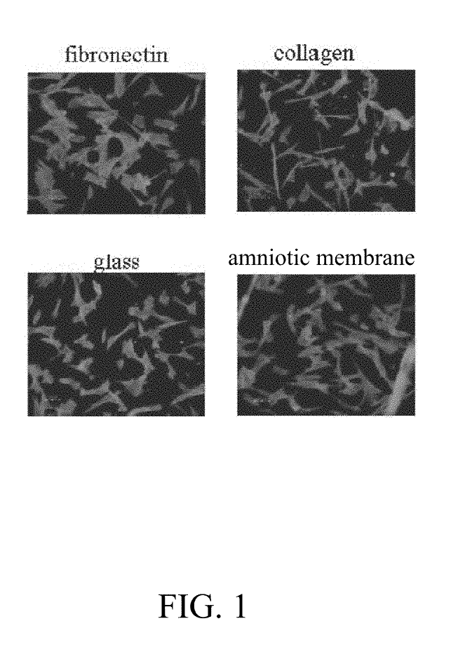

In a separate experiment, adherent placental stem cells' culture characteristics on dried amniotic membrane were compared to culture characteristics on fibronectin, collagen or glass. Coverglass surfaces were adsorbed with 10 μg/mL fibronectin or 500 μg/mL VITROGEN™. Dried amniotic membrane was secured to the bottom of 24 well plates with silicone rings. Placental stem cells were cultured at approximately 1×10⁴ cells/cm² on fibronectin, VITROGEN™, glass or amniotic membrane for 24 hours. Cells were then fixed and stained for the actin cytoskeleton. The results showed that placental stem cell spreading on amniotic membrane was very similar to that observed on fibronectin-coated surfaces. Placental stem cells also spread on cell culture-treated coverglass and collagen, though the placental stem cells appeared to display more thin, elongated cells in comparison. See FIG. 1.

Conclusions.

The dynamic structural rearrangement of the intracellular actin cytoskeleton is fundamental to many cellular activities such as adhesion, migration, and proliferation, and the extracellular milieu in which reside plays a role in regulating these cellular behaviors. Cultured fibroblasts and placental stem cells on amniotic membrane showed cellular spreading dynamics similar to cells cultured on fibronectin, a matrix protein known to be optimal for cell attachment and growth. This suggests that, although the amniotic membrane is composed mainly of collagen, other minor components may also be influencing cell adhesion and proliferation. These results also suggest that cells used in this study, both undifferentiated and terminally differentiated, find amniotic membrane a suitable scaffold for attachment and for functionality.

### 6.7. Example 7: Differentiation of Placental Stem Cells on Amniotic Membrane

This Example demonstrates osteogenic differentiation of placental stem cells on dried amniotic membrane.

### Materials

Osteogenic differentiation medium: DMEM low glucose supplemented with 10% fetal bovine serum (FBS) and 1×P/S+50 uM Ascorbic Acid+100 nM Dexamethasone+10 mM Beta Glycerol Phosphate (BGP).

Basal medium: DMEM low glucose with 10% FBS and 1×P/S.

Alternative carbon source medium: DMEM low glucose with 10% FBS and 1×P/S+10 mM BGP was used to see if these cells are inducible with phosphate source alone.

### Methods

Sterile dehydrated amniotic membrane cut to approximately the size of a single well (about 1.5 cm diameter) from a 6×8 cm sheet. The amniotic membrane piece was held in place by a silicone O-ring was cut to the size of the well (about 1.5 cm diameter). Placental stem cells, bone marrow-derived stem cells (BMSCs), and normal human dermal fibroblast (NHDF) cells were seeded onto the membrane at about 10000 cells/cm² in DMEM with 10% FBS and 1×P/S. The cells were allowed to grow over 2-3 days at 37° C. in 5% CO₂. The medium was then switched to differentiation medium, basal medium, or basal medium comprising beta glycerol phosphate (BGP). Differentiation was allowed to proceed for 21 days. Media was changed every 2-3 days.

Histological staining using the Mallory-Heidenhain stain technique was used to assess osteogenic differentiation. See James E. Dennis egt al., “In Vivo Osteogenesis Assay; a Rapid Method for Quantitative Analysis,” Biomaterials 19:1323-1328 (1998). Briefly, stem cell cultures were fixed in 4% paraformaldehyde, and embedded in paraffin. Sections 5 μm thin were cut from the paraffin block onto glass slides.

Preparation of Stain Solutions. 0.5% Acid Fuchsin: 0.5 g of Acid Fuchsin in 100 mL distilled water. Aniline Blue Solution: 0.5 g Aniline Blue, 2 g Orange G, 1 g phosphotungstic acid, dissolved in 100 mL distilled water. All reagents were from Sigma-Aldrich.

Procedure: Sections were deparaffinized using xylene and rehydrated through graded ethanol. Sections were then rinsed with distilled water and stained. Sections were first stained in Acid Fuchsin solution for 5 min. Excess dye was wiped from the slides, and the slides were immersed in Aniline Blue solution for 1 hour. Slides were transferred to 95% ethanol in several changes to remove excess dye. Sections were dehydrated clear and mounted with a synthetic resin.

### 6.8. Example 8: Preparation of Collagen Biofabric Comprising Crosslinked Hyaluronic Acid

This example demonstrates the production of a composite collagen biofabric comprising a crosslinked hyaluronic acid coating for use in culturing stem cells, e.g., placental stem cells. Materials and methods.

Collagen biofabric was provided as a dehydrated (less than or equal to 20% water) decellularized amniotic membrane. Hyaluronic acid (Fluka BioChimika) was provided as a 10 mg/mL solution in ultrapure water. Crosslinking agents used were 1,4-butanediol diglycidyl ether (BDDE; Sigma Aldrich), 1-ethyl-3-(3-dimethylaminopropyl)carbodiimide hydrochloride (EDCI; Sigma Aldrich), or divinyl sulfone (Fluka).

### Hyaluronic Acid Composites.

Hyaluronic acid is a glycosaminoglycan that is readily available, inexpensive and biocompatible, and which has good water retention and rheological properties.

Two different hyaluronic acid crosslinking methods were evaluated. Hyaluronic acid was crosslinked using either BDDE or divinyl sulfone using solution crosslinking, which involves combining hyaluronic acid with the respective crosslinker in solution and stirring overnight. Hyaluronic acid was also crosslinked using EDCI by immersion crosslinking, in which the hyaluronic acid was prepared as a solid film or foam, followed by immersion of the composition in a solution comprising the crosslinker.

Hyaluronic acid solutions were made using ultrapure water. Initially, it was determined that if the pH is too high, crosslinking will not occur, but that crosslinking proceeded without problems in ultrapure water.

Two-mL solutions of hyaluronic acid (10 mg/mL) were prepared and crosslinked with either BDDE (in solution; 2 μL/mg hyaluronic acid) or EDCI (by the immersion technique; 15 mM EDCI in 80:20 EtOH:water). Both techniques were successful at producing crosslinked films. Visually, the BDDE-crosslinked film appeared to swell much more than the EDCI-crosslinked film, indicating that the EDCI-crosslinked film contained more crosslinks than the BDDE-crosslinked film, an evaluation confirmed by differential scanning calorimetry (DSC) analysis. According to FTIR analysis, essentially no crosslinker remained in the hyaluronic acid films.

Due to the large amount of swelling noted with the BDDE crosslinked film, the crosslink density was increased. Samples were prepared with 1, 2 or 4 μL crosslinker per milligram of hyaluronic acid in solution. Crosslinking was performed at pH 5 or pH 7. In each case, solutions were crosslinked overnight and were lyophilized to produce sponge-like foams. Foams produced in 4 μL BDDE per milligram hyaluronic acid were very fragile and light, and when placed in water, they crumbled into mush. the other combinations produced foams that had acceptable structure and which swelled considerably in water. Neither the pH nor the amount of BDDE appeared to make a difference on the equilibrium water content (93% to 98%) or on the structure as determined by FTIR.

Several strategies for combining hyaluronic acid and dried amniotic membrane were attempted. Initially, a crosslinked hyaluronic acid solution was prepared, and 1 mL was placed on a section of dried amniotic membrane held in a frame that prevented movement of the membrane and leakage of the solution. The composite was air-dried and good attachment was noted. However, when placed in water, the amniotic membrane and hyaluronic acid separated.

In a second strategy, I mL of the hyaluronic acid solution was placed on to of amniotic membrane, and the composite was lyophilized. Following drying, the composite was immersed in an EDCI solution in 80:20 EtOH:water. However, when re-lyophilized, the HA pulled off of the amniotic membrane. When air-drying was substituted for the second lyophilization, a tight bond formed between the amniotic membrane and hyaluronic acid. When placed in water, the hyaluronic acid swelled without separating from the membrane.

The collagen biofabric, comprising hyaluronic acid, can be used as described herein to culture stem cells, e.g., placental stem cells, e.g., CD34⁻ placental stem cells.

### EQUIVALENTS

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

1. A method of culturing a stem cell comprising culturing the stem cell in a culture medium with a collagen biofabric, wherein said collagen biofabric is derived from a placenta, and said stem cell is exogenous to said collagen biofabric.

2. The method of claim 1 wherein said collagen biofabric comprises an amniotic membrane and/or a chorion isolated from a mammalian placenta.

3.-4. (canceled)

5. The method of claim 1, wherein said collagen biofabric is substantially dry prior to said culturing.

6. The method of claim 1, wherein said collagen biofabric is decellularized prior to said culturing, or wherein said collagen biofabric is not decellularized prior to said culturing.

7. (canceled)

8. The method of claim 1, wherein said collagen biofabric further comprises cells endogenous or exogenous to a placenta from which the collagen biofabric is derived.

9. (canceled)

10. The method of claim 8, wherein said collagen biofabric is irradiated.

11.-13. (canceled)

14. The method of claim 1, wherein said stem cell is an embryonic stem cell, a placental stem cell, a mesenchymal stem cell, a hematopoietic stem cell, a placental blood- or umbilical cord blood-derived stem cell, a bone marrow-derived stem cell, or an adult somatic stem cell.

15. The method of claim 14, wherein said adult somatic stem cell is a neural stem cell, a hepatic stem cell, a pancreatic stem cell, an endothelial stem cell, a cardiac stem cell, or a muscle stem cell.

16. The method of claim 1, wherein said stem cell is cultured for 24 hours or more, two days or more, or seven days or more.

17.-18. (canceled)

19. A method of differentiating a stem cell comprising culturing said cell in culture medium with a collagen biofabric for a time sufficient for differentiation of the cell.

20. The method of claim 19, wherein said collagen biofabric comprises an amniotic membrane and/or a chorion isolated from a mammalian placenta.

21.-22. (canceled)

23. The method of claim 19, wherein said collagen biofabric is substantially dry prior to said differentiating.

24. The method of claim 19, wherein said collagen biofabric is decellularized prior to said differentiating, or wherein said collagen biofabric is not decellularized prior to said culturing.

25. (canceled)

26. The method of claim 19, wherein said collagen biofabric further comprises cells endogenous or exogenous to a placenta from which the collagen biofabric is derived.

27. (canceled)

28. The method of claim 19, further comprising culturing a somatic cell on said collagen biofabric.

29.-30. (canceled)

31. The method of claim 19, wherein said stem cell is an embryonic stem cell, a placental stem cell, a mesenchymal stem cell, a hematopoietic stem cell, a placental blood- or umbilical cord blood-derived stem cell, a bone marrow-derived stem cell, or an adult somatic stem cell.

32. The method of claim 31, wherein said adult somatic stem cell is a neural stem cell, a hepatic stem cell, a pancreatic stem cell, an endothelial stem cell, a cardiac stem cell, or a muscle stem cell.

33. The method of claim 19, wherein said cell is differentiated into a neural cell, an adipocyte, a chondrocyte, an osteocyte, a hepatocyte, a pancreatic cell, or a cardiac cell.

34. A method of determining the toxicity of a compound to a cell, comprising culturing said cell with a collagen biofabric under conditions suitable for the survival of the cell; contacting said cell with the compound; and identifying a change in a metabolic parameter of said cell indicating apoptosis, necrosis, or cell death, or a tendency towards apoptosis, necrosis or cell death, as compared to a cell cultured under equivalent conditions and not contacted with said compound, wherein if said change is identified, said compound is toxic to said cell.

35. (canceled)

36. The method of claim 1, wherein said collage biofabric comprises hyaluronic acid, or wherein said collage biofabric comprises hyaluronic acid and wherein said hyaluronic acid is crosslinked to said collagen biofabric.

37. (canceled)
